(19) United States
(12) Patent Application Publication
(10) Pub. No.: US 2018/0306819 A1
(43) Pub. Date: Oct. 25, 2018

(54) ANTI-PLATELET RESPONSE AND REACTIVITY TEST USING SYNTHETIC COLLAGEN

(71) Applicant: JNC CORPORATION, Tokyo (JP)

(72) Inventor: William M. TROLIO, Horsham, PA (US)

(21) Appl. No.: 15/880,273

(22) Filed: Jan. 25, 2018

Related U.S. Application Data
(62) Division of application No. 14/420,597, filed on Feb. 9, 2015, now abandoned.
(60) Provisional application No. 61/681,485, filed on Aug. 9, 2012.

(30) Foreign Application Priority Data
Jul. 5, 2013 (US) PCT/US2013/049418
Aug. 5, 2013 (US) PCT/US2013/053612

(51) Int. Cl. G01N 33/86

(52) U.S. Cl. CPC G01N 33/86; G01N 2800/52; G01N 2800/222; G01N 2333/78

(57) ABSTRACT

The present invention provides platelet aggregation tests using synthetic, self-assembling type I human collagen, methods of measuring an individual's platelet anti-platelet medication sensitivity and residual platelet activity status when the individual is on a an anti-platelet medication and kits useful in the assays and methods.

This application is a divisional of U.S. patent application Ser. No. 14/420,597 filed on Feb. 9, 2015 (pending), which is a U.S. National Phase under 35 U.S.C. § 371 of International Application PCT/US2013/054078, filed on Aug. 8, 2013, which claims priority to U.S. Provisional Application 61/681,485, filed on Aug. 9, 2012, which are incorporated herein in their entirety. This application also claims priority to PCT applications, PCT/US2013/049418, filed on Jul. 5, 2013 and PCT/US2013/053612, filed on Aug. 5, 2013. All publications, patents, patent applications, databases and other references cited in this application, all related applications referenced herein, and all references cited therein, are incorporated by reference in their entirety as if restated here in full and as if each individual publication, patent, patent application, database or other reference were specifically and individually indicated to be incorporated by reference.

### BACKGROUND OF THE INVENTION

The conventional, primary need for an effective assessment of platelet response and reactivity is in the field of cardiology. The public health incidence and burden of heart attack, stroke and related cardiovascular and thrombotic diseases are well known. Increasingly, other fields of medicine such as orthopedics are incorporating the use of antiplatelet therapies to improve patient outcomes but do not have the necessary laboratory support and guidance. The medical community has long recommended the use of aspirin in primary care to reduce cardiovascular, stroke and certain other risks. New formulations of aspirin, aspirin in combination with other drugs and ‘super’ aspirins are currently under development and will expand the use of aspirin through many specialties. Aspirin (salicylate based compounds) ingestion or exposure inhibits the COX 1 pathway and modifies COX 2 enzymatic processes, which then precludes all subsequent events necessary for platelet aggregation. Since the ingestion of, or exposure to, aspirin can inhibit platelet aggregation, it has been given as a therapy to prevent undesired platelet aggregation, which can be a source of many heart attacks strokes and other thrombotic or bleeding conditions. Despite the benefits of aspirin therapy in many individuals, aspirin therapy is not effective enough in some individuals because the residual platelet reactivity is high and thus the patient's risk is not mitigated. Thus, physicians often prescribe an anti-platelet medication to patients whom are in need of a treatment to inhibit unwanted platelet aggregation.

Not all patients will respond to the anti-platelet medication in the same way, so there is a need for a reliable tool to assess and manage the anti-platelet medication response and reactivity on platelet aggregation. There currently is not an effective way to measure a patient's response to an anti-platelet medication therapy or to determine the patient's residual platelet reactivity. Thus, there is an unmet need for a reliable tool to assess and manage the anti-platelet medication response and reactivity on platelet aggregation when a patient is on an anti-platelet medication therapy, as well as checking patient compliance with the treatment regimen. Thus, there remains a need for a platelet activity test that does not use an animal derived collagen as the agonist and is able to measure the platelet response to an anti-platelet medication. The present invention meets this need.

It is widely believed that anti-platelet therapy contributes to the reduction of major atherothrombotic complications in cardiovascular, neurovascular and other diseases however, outcomes have not been predictable. In the treatment of percutaneous coronary intervention and acute coronary syndromes, dual anti-platelet therapy when performed at optimal dosing and timing has significantly lowered the risk of thrombotic complications and contributed to positive outcomes. However, an important clinical problem relates to the variability in patient response to anti-platelet treatments, the incidence of major adverse clinical events (“MACE”) and confounding differences in patient outcomes. Understanding the mechanisms underlying this phenomenon is important to improving patient care, long term (maintenance) therapy and consistent (positive) outcomes. However, a clear and reliable predictive model for responsiveness to anti-platelet therapy is currently not available. Attempts have been made to characterize the efficacy of anti-platelet therapy using platelet function testing but based on current information, its routine use is not recommended particularly as costs, complexities and cost effectiveness have not been established, and lack of correlation, standardization and agreement between laboratory methods is well documented in the literature. Tobias Geislera et al., Circulation 2010; 122:1049-1052. In addition, the inhibitory effects of aspirin on platelets decreases over time in patients on long term or chronic therapy. Violi, F. et al., J Am Cardio, Vol. 43, No. 6, 2004.

Thus, there remains a need for a quantitative, functional platelet activity test that does not use an animal derived collagen as the agonist. There remains a need for a test that is able to measure the platelet response to an anti-platelet medication when the patient is on a dual therapy of aspirin and an anti-platelet medication, as well as a test to monitor patient compliance with the dual therapy regimen. There is also a need for a test that can measure residual platelet activity (that is the platelet activity that remains even while the patient is on a dual anti-platelet medication therapy). The present invention meets these needs.

Traditionally, a patient's response to aspirin and other anti-platelet medication therapy is assessed by testing platelet activity with a platelet aggregation test. The “gold standard” of platelet aggregation tests, light transmission aggregometry (LTA), utilizes collagen from biological sources as the agonist to bring about platelet aggregation, as a measure of the degree or extent of platelet response or inhibition to aggregation. However, there are multiple issues as well as the risk of infectious disease transmission when using biological material. Biologically derived products, whether ‘natural,’ processed, manufactured by fermentation, cell culture or similar processes, or recombinant, all share the following drawbacks: carry a risk of infectious disease transmission; have lot to lot variability (regarding the ratio of active materials, performance, chemical characteristics, solubility, stability, moisture content, and process contaminants); differing bio-profiles depending upon the location the product was made; differences caused by processing; and environment, geographic and dietary differences affecting the source animal or culture.

There are 29 (currently) identified types of collagen. Fibrillar collagens include types I-III, V and XI, recently characterized types XXIV and XXVII. (Esposito, J Y et al, The fibrillar Collagen Family. Int J Mol Sci. 2010; 11(2): 407-426.) Typically, type 1 fibrillar collagen is preferred for platelet aggregation, although the use of types IV and III have been reported. All 29 available collagens are from biological sources (calf skin, acid extracted, fractionated calf skin, bovine tendon, bovine nasal septum, equine tendon, burro aorta, rabbit aorta, rat skin, rat tail, mouse sternum, kangaroo tail, recombinant (nicotania), human placenta, and human lung). There is no single collagen product standard or standard collagen product. The source of collagen, its preparation, and its concentration each contribute additively to the variability of platelet response to collagen. This variability makes the use of biological collagen for precise therapeutic control of collagen sensitive anti-platelet agents unobtainable.

It is widely believed that anti-platelet therapy contributes to the reduction of major atherothrombotic complications in cardiovascular, neurovascular and other diseases. In the treatment of percutaneous coronary intervention and acute coronary syndromes, anti-platelet therapy when performed at optimal dosing and timing has significantly lowered the risk of thrombotic complications and contributed to positive outcomes. However, an important clinical problem relates to the variability in patient response to anti-platelet treatments, the incidence of major adverse clinical events (“MACE”) and confounding differences in patient outcomes. Understanding the mechanisms underlying this phenomenon is important to improving patient care, long term (maintenance) therapy and consistent (positive) outcomes. However, a clear and reliable predictive model for responsiveness to anti-platelet therapy is currently not available. Attempts have been made to characterize the efficacy of anti-platelet therapy using platelet function testing but based on current information, its routine use is not recommended particularly as costs, complexities and cost effectiveness have not been established, and lack of correlation, standardization and agreement between laboratory methods is well documented in the literature. Tobias Geislera et. al., Circulation 2010; 122:1049-1052. In addition, the inhibitory effects of aspirin on platelets decreases over time in patients on long term or chronic therapy. Violi, F. et al., J Am Cardio, Vol. 43, No 6, 2004.

Thus, there remains a need for a quantitative, functional platelet activity test that does not use an animal derived collagen as the agonist. There remains a need for a test that is able to measure the platelet response to an anti-platelet medication, as well as a test to monitor patient compliance. There is also a need for a test that can measure residual platelet activity (that is the platelet activity that remains even while the patient is on an anti-platelet medication therapy). The present invention meets these needs.

### SUMMARY OF THE INVENTION

The present invention provides tests for determining an individual's donor's anti-platelet medication sensitivity status when the individual is on an anti-platelet medication therapy comprising the use of synthetic collagen. Exemplary tests include the use of platelet aggregation studies using for example, light transmission aggregation assays (LTAAs) and flow cytometry. The tests of the present invention use synthetic collagen as the agonist. Other tests, including impedance aggregation and related technologies are also contemplated.

The test and methods of the present invention are able to test the ability of the individual's platelets to aggregate after the individual has ingested an anti-platelet medication. In these embodiments the final in-test concentration of synthetic collagen used preferably ranges from about 2.0 ng/mL to about 500 ng/mL.

In another embodiment, the present invention provides methods for determining an individual's anti-platelet medication sensitivity status (which may be hypersensitivity, average sensitivity, or non-responder or hyposensitive).

The present invention also provides a method of testing patient compliance, with the anti-platelet therapy regimen.

The present invention also provides tests that help predict the effectiveness of an anti-platelet medication for a patient.

Certain embodiments of the present invention utilize synthetic collagen at amounts at least 1000 fold less than similar assays using biological collagen.

In certain embodiments, the synthetic collagen is a synthetic collagen that has the ability to self-assemble into a triple helix to form fibrils and which mimics human type I collagen. In certain embodiments the synthetic collagen comprises a polypeptide having a peptide fragment represented by the formula (I)

-(Pro-X-Gly)ₙ (I)

wherein X represents Hyp; and n represents an integer of from 20 to 5,000; and

wherein the polypeptide has a molecular weight at a range of from 10,000 to 500,000. In certain embodiments, n=20-250.

The present invention also provides kits for testing platelet aggregation in a light transmission assay, comprising a vial of synthetic collagen; and instructions for use of the synthetic collagen in the anti-platelet medication therapy test (APMTT) and assays of the present invention.

In certain embodiments (including the methods described herein and the kits), the synthetic collagen is supplied and/or stored in a polypropylene homomer container. In certain embodiments, the cap is the same material as the vial/tube. In certain embodiments, the container has an additional internal seal or a cap having a secondary seal molded therein. In certain embodiments, the container contains all of the above described characteristics

### BRIEF DESCRIPTION OF THE FIGURES

FIG. 1 shows the results of LTAAs run with biological collagen.

FIG. 2 provides the results of LTAAs run with synthetic collagen. This figure shows a normal response over a range of concentrations of synthetic collagen.

FIG. 3 provides the results of various LTAAs run with synthetic Collagen in response to several individual anti-platelet medications (Aggrenox®, Integrelin® and Reopro®).

FIGS. 4A-C show the variability of results obtained when one uses biologically derived collagen in LTAAs. All of the LTAAs run for FIGS. 4A-C were run on the same individual's platelet samples, on the same aggregometer, and same testing procedures and the collagen was obtained from the same vendor. Thus the variation in these tests is attributable to the variations in biological collagen. FIG. 4A shows variation measured by percentage aggregation. FIG. 4B shows variation measured by lag phase. FIG. 4C shows variation measured by area under the curve.

FIG. 5 shows the activation of platelets in citrated whole blood by various collagen reagents, including synthetic collagen (see example 1) as assessed with flow cytometry.

FIG. 6 shows the activation of platelets in citrated whole blood by various collagen reagents (see example 1) as assessed with flow cytometry.

FIGS. 7-8 show the results of tests where synthetic collagen was used to detect anti-platelet activity of various anti-platelet medications. In these tests, flow cytometry was used to measure platelet aggregation. See Example 1.

FIG. 9 shows the effect of ticagrelor on agonist-induced platelet aggregation.

FIG. 10 shows the effect of cilostazol on agonist-induced platelet aggregation.

FIG. 11 shows the effect of abciximab on agonist-induced platelet aggregation.

FIG. 12 shows biological collagen at 5 and 2 μg/mL. In using the whole blood mode of the Chrono Log aggregometer, a test measuring platelet aggregation on whole blood (impedance aggregation), at 5 μg/mL, the biological collagen gets a response. At 2 μg/mL, there is no response. As an aside, even though called “whole blood mode” by the manufacture, most often whole blood is actually diluted whole blood (usually a 1:1 or greater dilution).

FIG. 13 shows that synthetic collagen can be diluted from 100 ng/mL to 12.5 ng/mL and still elicit the same response using the whole blood mode of the Chrono Log aggregometer, a test measuring platelet aggregation on whole blood (impedance aggregation).

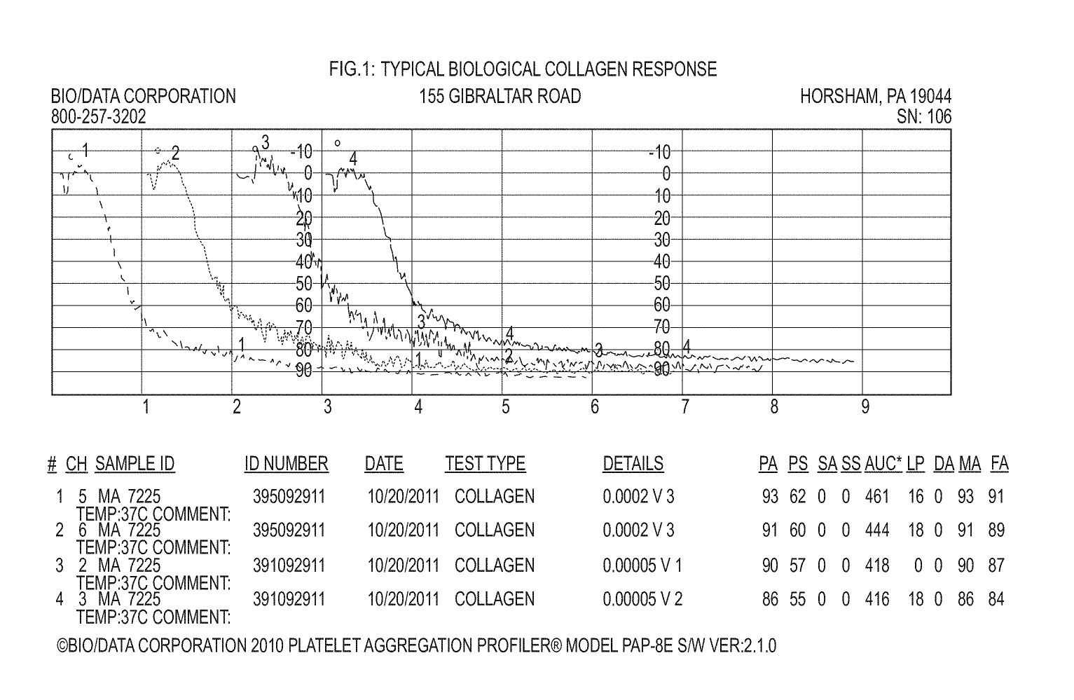

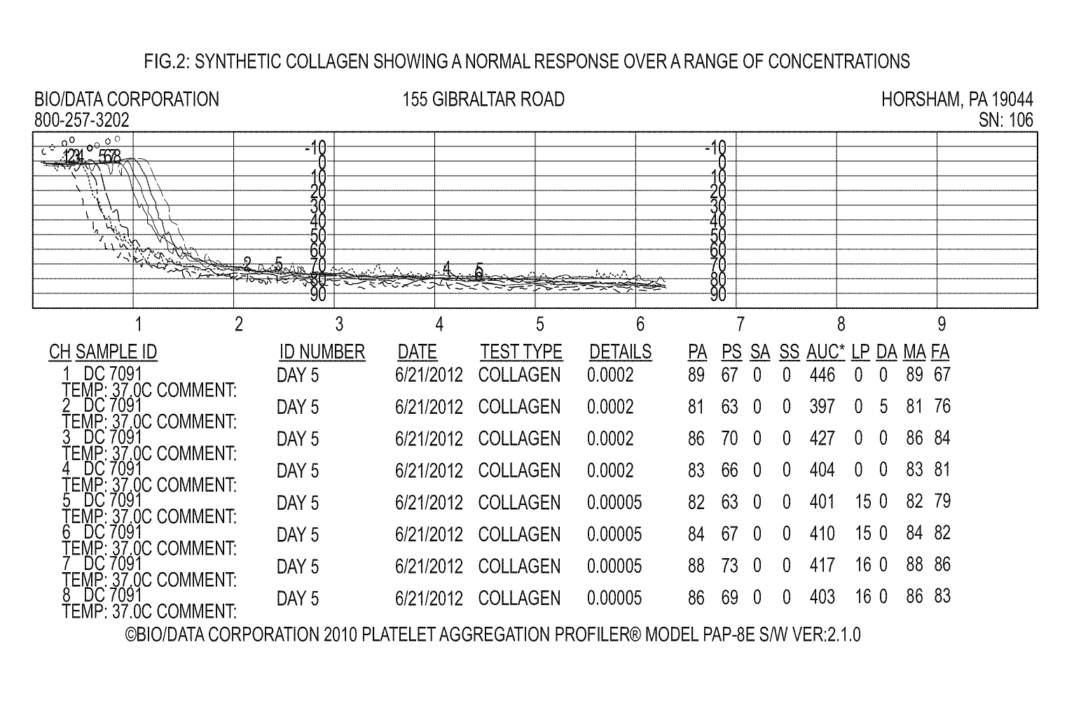

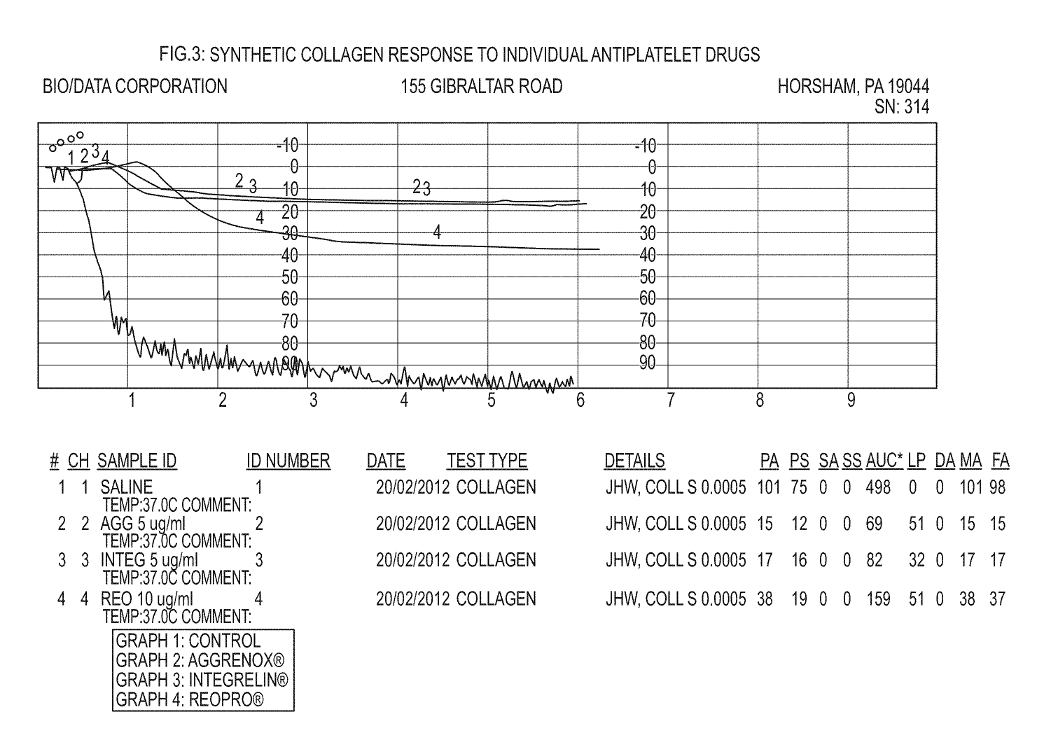

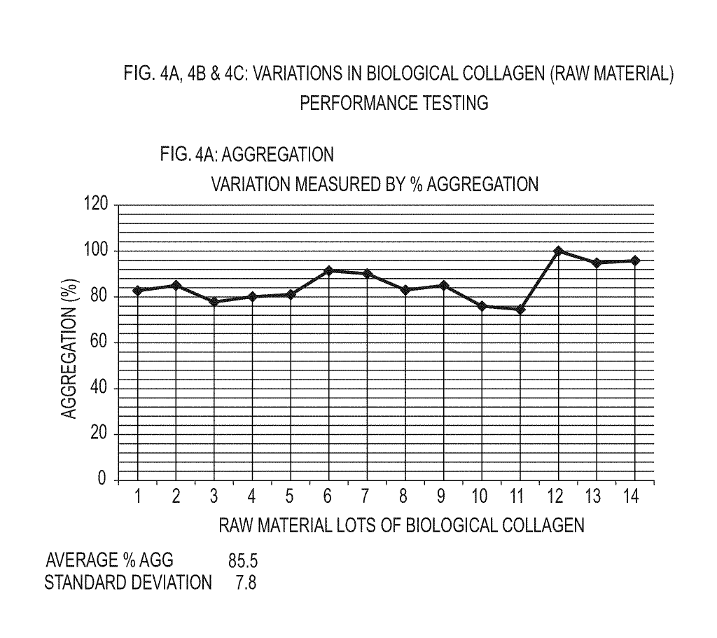

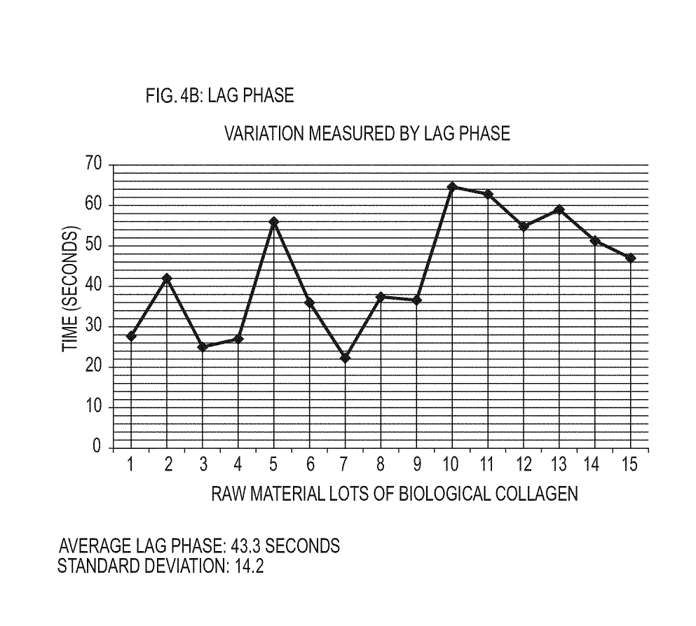

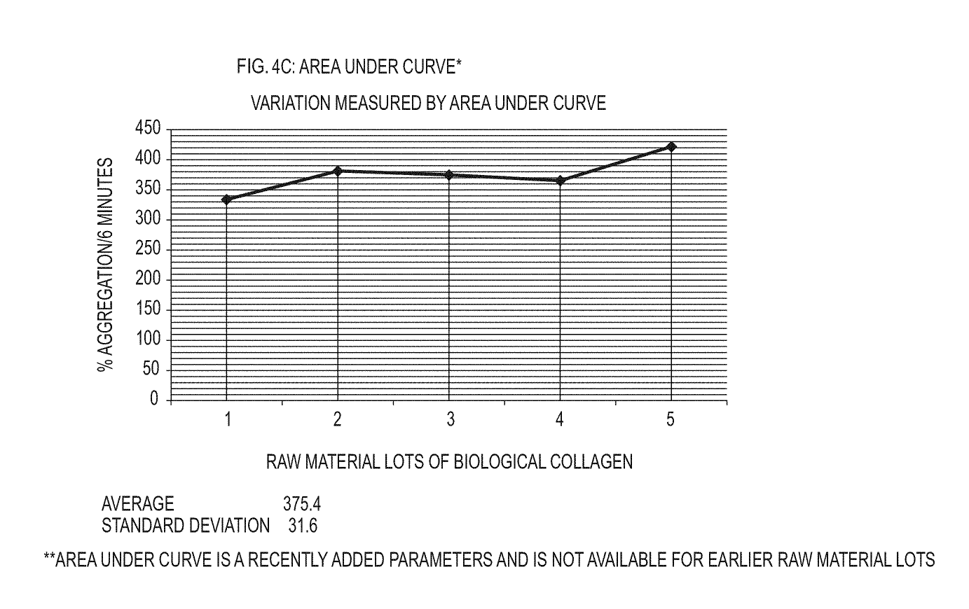

### DETAILED DESCRIPTION OF THE INVENTION

The present invention provides an anti-platelet medication therapy test (APMTT). The APMTT test is a unique, quantitative, functional test, based on different synthetic collagen concentrations, that measures the patient's response to different classes of anti-platelet medications, as well as the residual platelet reactivity of the anti-platelet medication inhibited patient platelets. The test results provide the physician with information about the patient's response to the anti-platelet medication as well as residual platelet activity that remains after exposure to the medication.

Residual platelet activity is the activity (functionality) of the platelets after they have been exposed to the anti-platelet medication therapy. No therapeutic dose will impair 100% of the platelets (nor would this be desirable). However, the reactivity of these non-impaired platelets is a key factor in understanding the individual's complete platelet response and MACE risk. For instance if the anti-platelet medication therapy were to impair 80% of the platelets (keeps 80% platelets from aggregation), there is still 20% of the individual's platelets that could cause thrombotic risk if they were to be extremely active or alternatively, could pose a risk of bleeding to death if the platelets were not active and would not aggregate (and thus would not clot).

The present invention provides a method for determining an individual's functional platelet response to an anti-platelet medication, wherein the anti-platelet medication does not include aspirin. Anti-platelet medications are known and include, but are not limited to, abciximab (Reopro®), anagrelide (Agrylin®), clopidogrel bisulfate (Plavix®), eptifabatide (Integrilin®), tirofiban (Aggrastat®), dipyridamole/aspirin (ASA) (Aggrenox®), cilostazol (Pletal®); dipyridamole (Persantine®), ticagrelor (Brilinta®), ticlopidine (Ticlid®), Aloxiprin (aluminum acetylsalicylate), Carbasalate calcium (mixture of calcium acetylsalicylate and urea), Cloricromen, Clorindione, Ditazole, Indobufen, Picotamide, Ramatroban, Terbogrel, Terutroban, and triflusal, as well as those in similar drug classes that are in various stages of development like cangrelor, elinogrel, prasugrel, and others. Anti-platelet medications are often classified based on their mode of action. For example, the table below provides exemplary classifications.

| Class | Example | Therapeutic Basis |
|---|---|---|
| 1 | Aspirin | Salicylate (COX Inhibitor) |
| | | IUPAC Name: 2-acetyloxybenzoic acid |
| 2 | Dipyridimole | Phosphodiesterase Inhibitor (cAMP and cAMP-inhibited cGMP 3′,5′- |
| | | cyclic phosphodiesterase 10A Activity Inhibitor) |
| | | IUPAC Name: 2-[[2-[bis(2-hydroxyethyl)amino]-4,8-di(piperidin-1- |
| | | yl)pyrimido[5,4-d]pyrimidin-6-yl]-(2-hydroxyethyl)amino]ethanol |
| 3 | Reopro ® | Not listed as Pubchem Compound |
| | | Immunoglobulin Fragment |
| | | Fab Fragment Chimeric monoclonal antibody 7E3 (GP IIbIIIa receptor |
| | | blocker) |
| 4 | Plavix ® | Thienopiryridine (PY2 receptor inhibitor) |
| | | IUPAC Name: methyl (2S)-2-(2-chlorophenyl)-2-(6,7-dihydro-4H- |
| | | thieno[3,2-c]pyridin-5-yl)acetate |
| 5 | Brilinta ® | Cyclopentyltriazolopyrimidine (P2Y₁₂ Inhibitor) |
| | | IUPAC Name: (1S,2S,3R,5S)-3-[7-[[(1R,2S)-2-(3,4- |
| | | difluorophenyl)cyclopropyl]amino]-5-propylsulfanyltriazolo[4, |
| | | 5-d]pyrimidin-3-yl]-5-(2-hydroxyethoxy)cyclopentane-1,2-diol |
| 6 | Aggregnox ® | Phosphodiesterase Inhibitor (cAMP-specific 3′,5′-cyclic |
| | | phosphodiesterase 4A Inhibitor) |
| | | IUPAC Name: 2-[[2-[bis(2-hydroxyethyl)amino]-4,8-di(piperidin-1- |
| | | yl)pyrimido[5,4-d]pyrimidin-6-yl]-(2-hydroxyethyl)amino]ethanol |

The methods of the present invention are able to test the ability of the individual's platelets to aggregate after the individual has ingested an anti-platelet medication, or in other words, test the ability of the anti-platelet medication to inhibit platelet aggregation in the presence of an agonist. In this case the agonist is synthetic collagen. The present invention may utilize multiple methods of measuring platelet aggregation including, but not limited to the use of light transmission aggregometry assays (LTAAs) (which uses platelet rich plasma (“PRP”); flow cytometry (which uses whole blood); and whole blood impedance aggregometry.

LTAA is the traditional (gold standard) assay used to test a patient's platelet aggregation in response to aspirin. The present inventors have determined that using synthetic collagen, LTAAs can be used to test a patient's platelet aggregation in response to an anti-platelet medication. In this assay, light is passed through a sample containing platelets and measured and compared to a platelet free plasma standard. The platelets are normally suspended in a solution so they will block a certain amount of light. Then a material (i.e. an agonist such as collagen) is added to the platelets to cause them to aggregate and again the light passing through the sample is measured. When the platelets aggregate (form clumps), more light will pass through the sample than when they are in a suspension. A comparison of the light measurement before and after the platelets aggregate informs the tester how much aggregation has occurred.

In most individuals, various anti-platelet medications will cause a variable degree of inhibition of platelet aggregation. These individuals are sometimes referred to as having an average/normal response or “low on therapy platelet reactivity.” However, in some individuals, even after taking the standard dose of anti-platelet medication, the platelets will still aggregate, and hence the particular anti-platelet medication would not be beneficial, but rather detrimental since the thrombotic risk would not be controlled, reduced or evident to the clinician. These individuals are often referred to being non-responsive. In such individuals, the clinician might change medication doses, try another drug class or even add a second drug to the anti-platelet regimen if the bleeding risk is reasonable. In yet other individuals, even very small doses of anti-platelet medication causes a severe inhibition of platelet aggregation that could lead to bleeding issues, which in some cases are life threatening or life ending. These individuals can be called hypersensitive. For these individuals a particular anti-platelet medication therapy may cause more harm than good because of the increased bleeding risk. Thus, it is very desirable to be able to test a patient/individual for their response to a particular anti-platelet medication to see what effects the anti-platelet medications will have on the patient's platelet aggregation and residual platelet reactivity to determine whether the anti-platelet medication therapy will be useful for preventing unwanted platelet aggregation or whether the particular anti-platelet medication therapy should be avoided because of bleeding, interference with other drug therapies, or other serious risks.

In addition, the tests can be used to monitor patient compliance in taking the prescribed anti-platelet medication. Compliance means taking the medication, and taking the medication at the proper time to maintain the risk-reducing effect of the anti-platelet medication. Non-compliance has been identified in multiple studies as a significant occurrence and carries a very high risk for the patient. Recently Medscape (July 25^{th}) reported that as many as half of all heart patients do not take their medications, thus confirming that effective compliance monitoring is a significant unmet medical need.

In embodiments of the invention utilizing LTAAs, the assay measures quantitatively multiple optical parameters from changes in light transmission through Platelet Rich Plasma (PRP) following the addition of an agonist (such as, collagen, ADP, epinephrine, Ristocetin, Arachidonic Acid, thrombin and TRAP) (e.g. more light passes through a sample where there has been platelet aggregation as compared to a sample with no platelet aggregation). An agonist is a material that when added to platelet rich plasma, causes the platelets to aggregate. In the present invention the agonist is synthetic collagen. In LTAAs, the PRP is usually stirred in a cuvette at 37° C., and the cuvette sits between a light course and a photocell (or solid state detectors). After an agonist is added to platelet rich plasma (PRP), the platelets aggregate and absorb less light, so the light transmission increases and is detected by the photocell.

LTAAs generate data in the form of aggregation patterns. The LTAA generates parameters plotted on an x/y grid. The x axis is usually a linear time base (typically—minutes). The y axis is a logarithmic scale based upon light transmittance. This light transmittance is equated to percent (%) aggregation.

As the LTAA pattern or curve is generated (primary data), various derived measurements are calculated, including slope (Sa); maximum aggregation; final aggregation; area under the curve (AUC), area under the slope (AUS) and others. In some embodiments of the present invention AUC is a preferred measurement aspect, because it appears more sensitive than the others. Slope of aggregation (Sa) is a measurement of the rate at which the reaction is proceeding. Dilution profile (DUP) is an incremental change in concentration of the reactants in a test mixture. In collagen testing, the DUP is comprised of the changes to the concentration of the collagen reagent used. Other dilution profiles may be defined and used in analyses. Slope of the dilution Profile (Sd) is generally the regression analysis of the change in concentration. Slope of the reaction profile (Sr) is generally the regression analysis of the change of reaction to change of dilution. The regression analysis may be linear, polynomial or other models. Area under the curve (AUC) is a receiver operating curve that is the calculated graphical volume from the start of the reaction to the end of the reaction as defined by the aggregation and slope of aggregation (Sa). The use of the AUC parameter increases the sensitivity of the DAPT assay(s). This may be considered as the “Power” generated by the reaction. In addition, lag phase (LP), disassociation (DA); and final aggregation (FA) are other readouts/parameters that may be used.

The present invention utilizes synthetic collagen, which is much more sensitive, potent, predictive and precise than biological collagen, and further is dilutable, which allows extremely low amounts of synthetic collagen to be used. Using synthetic collagen, the present invention is able measure the degree to which the patient's platelets resist aggregation after the patient has ingested an anti-platelet medication. This is a key element, which permits the clinician to accurately assess thrombotic or bleeding risk, and decide on the appropriate therapy. Further, synthetic collagen provides a means of quantitatively assessing residual platelet reactivity, which is the key indicator of prognostic risk, and is, therefore, more useful information than the currently available, qualitative and highly variable parameter called platelet inhibition. It is important to note that inhibition of aggregation does not equal residual platelet reactivity. Until recently, “platelet inhibition” was the global term and test parameter that was accepted for understanding how platelets behaved when exposed to an anti-platelet drug. The percent aggregation or percent inhibition of aggregation was simply adopted because that is how platelet aggregation was reported. So, inhibition was simply the difference between the patient's original aggregation result and the post treatment result. If the patient's original aggregation result was 82% and the post treatment aggregation result was, 23%, the percent inhibition was reported as 59%. It was then assumed that the other 41% of the platelets were not inhibited. The target was to get the percentage of inhibition between 60 and 80% because that would mean the patient would neither clot nor bleed (ideal outcome). However, it has now been determined that this simplistic construct or understanding does not tell the whole story and thus does not work because it does nothing to aid in the understanding of the residual platelet reactivity (that is, the reactivity of the platelets that did not respond to the ingested medicine). There simply is no direct, measurable or predictable relationship between percent inhibition and residual platelet reactivity.

Physicians have discovered that patient outcomes to various treatments varied even when they shared the same percent inhibition. For example, two patients who showed 41 percent inhibition (and thus had 59% aggregation) in an LTA might respond very differently (one may still have problems with unwanted clotting and the other may have problems with bleeding). This observation has slowly led to the realization that although the percent inhibition number (the number of remaining or uninhibited platelets) could be identical, the patient responses could be very different. This has recently spawned the use of terms such as hyper and hypoactive, hyper and hypo-responders, platelet reactivity, residual platelet reactivity, and High on Treatment Platelet Reactivity.

The point is that percent inhibition is not the same as residual platelet reactivity. It is clear that platelet response is the measurable effect of a challenge on platelet function, i.e., how a particular drug interferes with a platelet function pathway (platelet and metabolic genetics, drug action and pharmacokinetics, and other factors all come in to play for residual platelet reactivity). The present invention can measure residual platelet reactivity, which is a combination of two concepts wrapped up into basically a single measurement. The first component is a dose response based on the primary drug (e.g. an anti-platelet medication) to show what portion of the patient's platelets are rendered partially or non-functional based on the that particular patient's individual response to that drug and dose. The second component relates to how reactive the remaining platelets are. These platelets, like the ones that are inhibited, could be hyperactive, hypoactive or anywhere on the continuum between those two points to a different medicine.

In one aspect, the synthetic collagen is used at a concentration that tests the residual activity of the platelets after being exposed to the anti-platelet medication.

In this aspect, the concentration of synthetic collagen is a concentration falls between about 2.0 ng/mL to about 500 ng/mL; between about 8 ng/mL to about 500 ng/mL, or between about 25 ng/mL to about 500 ng/mL, or between about 50 ng/mL to about 500 ng/mL, or between about 50 ng/mL to about 250 ng/mL. The concentration may be about 8 ng/mL, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, . . . 490, 495 ng/mL, and up to an including 500 ng/mL.

There are ranges of platelet aggregation that occur after an individual consumes an anti-platelet medication and these ranges can be used to characterize an individual as having a hypersensitive response, a normal/average response, or having a non-response to the anti-platelet medication. Different individuals respond to a particular anti platelet medications differently, so the present invention provides a way of measuring the response to the anti-platelet medication as well as residual platelet reactivity. If the individual does not respond to the medication as desired, the physician can then change the dose, prescribe a different anti-platelet medication or even add a second anti-platelet medication or a low dose aspirin regimen.

The present invention also provides embodiments that capitalize on the repeatability and sensitivity of the synthetic collagen as well as its ability to be reproducibly diluted over a range of concentrations and thus, employs multiple dilutions of synthetic collagen (referred to herein as “dilution profiles”) to aid a physician in determining not only whether an individual is sensitive to the anti-platelet medication, but to further understand an individual's anti-platelet medication sensitivity status (e.g. the degree to which an individual is anti-platelet medication sensitive, non-responsive or hypersensitive). Dilution profiles, when presented graphically have distinctive shapes and changes that may be further visually assessed rapidly much like an EKG.

This information may be useful for the physician to determine an appropriate dose of anti-platelet medication and/or aspirin in the prescribed therapeutic regimen, or perhaps whether a second or third therapeutic medicine is required, or whether to consider abandoning the use of the anti-platelet medication altogether for an alternative therapy. It also allows the physician to monitor the drug's effect over time and patient compliance. The effectiveness of certain drugs such as aspirin, when used chronically, decrease over time. This is another reason for periodic monitoring tests using the method of the present invention, which use synthetic collagen.

Further capitalizing on the sensitivity of synthetic collagen and further using the dilution profile concept, the present invention also provides embodiments where an individual's anti-platelet medication sensitivity can be predicted even before the individual ingests the anti-platelet medication. Anti-platelet medication non-responder (resistant) individuals have a distinct response (referred to herein as the “bounce back”) to LTAAs run over varying concentrations of synthetic collagen, which can be used to diagnose an individual's response to the anti-platelet medication. This and other embodiments are discussed more fully herein below.

As mentioned above, LTAAs use Platelet Rich Plasma (PRP), which is prepared from properly anti-coagulated whole blood. The individual's blood is collected and spun down to obtain the PRP. Since platelets are very sensitive and can be readily activated during the preparation of PRP, the individual's blood is usually collected in a tube containing a particular anticoagulant. For example, venous blood is obtained and collected into 3.2% sodium citrate in a ratio of 1:9 (1 part anticoagulant to 9 parts blood). Whole blood samples should be processed within 4 hours of collection and blood samples for platelet aggregation testing must be stored at room temperature as cooling the platelets can lead to activation and erroneous test results. PRP is usually prepared by centrifugation at 20° C. for 10-15 minutes at 150-200 g. The PRP is carefully removed and placed into a capped plastic tube. PRP must be stored at room temperature.

Platelet poor plasma (PPP) can be then prepared by further centrifugation of the remaining plasma at 2700 g for 15 minutes. Platelet poor plasma (PPP) contains no platelets or other cellular material, and is often used as a blank in LTA sample analyses. In certain embodiments a special centrifuge, called a platelet function centrifuge or PDQ®, that can generate PRP and PPP in about 5 minutes instead of the typical 45-60 minutes is employed. This makes the LTAA even more practical for emergency and critical care situations or for a high throughput clinical setting. In addition to the rapid sample preparation, the use of certain concentrations of synthetic collagen can be used to generate the patient's global residual platelet reactivity on an emergency basis.

Addition of a platelet agonist to the PRP usually leads to platelet activation, and leads to a change in their shape from discoid to spiny spheres, which is associated with a transient increase in optical density. Exceptions to this are epinephrine in which there is no shape change, and ristocetin, which causes platelet agglutination rather than aggregation, i.e. there is no binding of fibrinogen. Agonists are usually classified as strong agonists or weak agonists. As examples, strong Agonists (e.g. Collagen, thrombin, TRAP, high concentration ADP, and U46619 (an analog of TxA2)) directly induce platelet aggregation, TxA2 synthesis and platelet granule secretion. Weak Agonists (e.g. low concentration ADP & epinephrine) induce platelet aggregation without inducing secretion.

In general, LTAs are performed at 37° C. The aggregometer is calibrated by: 1) a cuvette containing PRP, which equates to 0% light transmission; and 2) a second cuvette containing PPP, which equates to 100% light transmission. Since platelets will normally only aggregate if they are activated (with an agonist—chemical or physical) and in contact with each other, they must be stirred whilst testing is taking place. Absence of stirring will lead to an absence of, or at least a significant reduction in, aggregation, producing an erroneous test result.

The present invention utilizes synthetic collagen as the agonist instead of collagen obtained from biological sources in the platelet aggregation tests, including light transmission aggregometry assays (“LTAA”). The use of synthetic collagen provides many unexpected benefits over the use of biological collagen, which are described herein.

In certain embodiments, Bio/Data's PAP 8E Light transmission aggregometer is employed (See U.S. Pat. No. 7,453,555) in the tests of the present invention that utilize LTAAs to measure platelet aggregation.

Normally a test for spontaneous platelet aggregation (“SPA”) is performed. SPA is rare in healthy individuals, but seen in people with hyperactive platelets and is also recognized as an independent marker for some pro-thrombotic conditions, in some cases of von Willebrand Disease (vWD), in some patients with diabetes, in some lipid disorders and in a variety of other disorders. The presence of SPA can be tested by placing undiluted PRP in the aggregometer and stirring for 15 minutes. In cases of SPA, dilution of the PRP with PPP or saline may abolish this and if the platelet count remains >200×10⁹/L then aggregation testing can proceed.

In general, about 225 μL of PRP is added to the aggregometry test cuvette and warmed at 37° C. Then 25 μL of the agonist is added and the response recorded. The typical readouts or responses recorded include primary aggregation (“PA”) (which usually provides a value indicating the amount of aggregation), primary slope (“PS”)(which usually provides a value relating to the rate of aggregation) and area under the curve (“AUC”)(which generally provides a value relating to a combination of PA and PS), lag phase, disaggregation and final aggregation. Aggregometers used in the field typically will provide some or all of these readouts along with a pictorial graph of the aggregation. Each aggregometer or system calculates the values a bit differently and may use a proprietary formulae embedded in the system's software.

For example, % maximal aggregation may be calculated by measuring the distance between the baseline [0% aggregation−platelet rich plasma] and platelet poor plasma [100% aggregation] [Y] and dividing this number by the maximal aggregation [X]. So if the Y=100 mm and X=89 mm then percentage maximal aggregation=X/Y=89%.

In embodiments of the invention that utilize multiple platelet aggregation assays, it is preferable that the same type of aggregation assay is used. For example, if the first test measures platelet aggregation with Platelet Rich Plasma and LTAAs, then each subsequence test also preferably measures platelet aggregation using the same LTAAs. Similarly, if the first test measures platelet aggregation using whole blood and flow cytometry, then each subsequent test also preferably measures platelet aggregation using flow cytometry.

### A1. Testing for Platelet Sensitivity to Anti-Platelet Medication in an Individual (Using Pre and Post Medication Readouts)

One embodiment of the present invention provides assays that can determine an individual's platelet sensitivity to an anti-platelet medication. This involves performing one or more platelet aggregation assays, such as LTAAs or the use of flow cytometry to measure aggregation, whereby a first platelet rich or whole blood sample is obtained from an individual and is combined with synthetic collagen to form a first treated sample. The final in-test concentration of synthetic collagen used for this embodiment and other embodiments described in the application ranges from about 2 ng/mL to about 500 ng/mL; from about 8 ng/mL to about 500 ng/mL; from about 10 ng/mL to about 500 ng/mL; from about 15 ng/mL to about 500 ng/mL; from about 20 ng/mL; from about 25 ng/mL to about 500 ng/mL; from about 30 ng/mL to about 300 ng/mL; from 40 ng/mL to about 400 ng/mL; from about 50 ng/mL to about 500 ng/mL; from about 8 ng/mL to about 100 ng/mL; from about 8 ng/mL to about 80 ng/mL; from about 8 ng/mL to about 50 ng/mL. Preferably the amount of synthetic collagen is at least about 8 ng/mL or at least about 2 ng/mL. These values and ranges are preferably used when the platelet aggregation tests are light transmission assays and the sample is a PRP sample that is tested. However, they may also be used in other analyzers, including flow cytometers and impedance aggregometers or their equivalents.

For the first treated sample, the individual has not ingested the anti-platelet medication for a time period of about 24 hours, preferably 72-96 hours. The idea is to make sure that the individual will not have any anti-platelet medication in his system to affect the platelet aggregation tests. The sample is measured for platelet aggregation to obtain a first readout to determine the individual's baseline level in the absence of ingested anti-platelet medication.

In certain embodiments, an initial platelet aggregation assay may be performed to check for spontaneous aggregation to test for whether the platelets have any inherent hyperactivity. Saline is added to the LTAA instead of the synthetic collagen to see if there is any aggregation.

Then the individual is given the anti-platelet medication and a time period sufficient to allow the anti-platelet medication to be metabolized (e.g. at least about 2 hours to about 16 hours) is allowed to pass before a second platelet rich plasma sample or whole blood sample is obtained from the individual. A platelet aggregation assay is performed on the second platelet rich plasma sample or whole blood sample by treating it with synthetic collagen to form a second treated sample. The sample is measured to obtain a second readout.

It is preferred that the same type of platelet aggregation assay is used throughout the process. For example, if LTAA is used for the first treated sample, then preferably LTAA is used for the second treated sample.

The baseline level readout in the absence of ingested anti-platelet medication is compared with the second treated sample readout (obtained after the anti-platelet medication ingestion) and the results of this comparison will determine the individual's anti-platelet medication response status. For example, if the individual shows a significant reduction in platelet aggregation after the anti-platelet medication ingestion (in the second sample) as compared to the baseline sample, then the individual may be characterized as normal or having an average anti-platelet medication sensitivity. If the individual shows very little difference in the platelet aggregation after taking the anti-platelet medication (i.e. the platelets still aggregated after the individual ingested the anti-platelet medication), then the individual may be characterized as being non-responsive to anti-platelet medication. If the individual showed an almost complete lack of platelet aggregation after ingesting the anti-platelet medication, then the individual may be characterized as being hypersensitive to the anti-platelet medication, which itself is a very high risk state for the patient (bleeding risk).

In the case of using LTAAs to measure platelet aggregation, the readout from the LTAA may be preferably the area under the curve, slope, primary aggregation, lag phase (LP), disaggregation (DA), final aggregation (FA), or a combination thereof. In certain embodiments the preferred readout is area under the curve.

For example, when using the Bio/Data's PAP 8E aggregometer, and when using an “in-test” concentration of 50 ng/mL of synthetic collagen, for an anti-platelet medication hypersensitive individual, the baseline for PA will range from 50% to 100%. The baseline for PS will range from 25 to 60. The baseline for AUC will range from 300 to 800. After the anti-platelet medication, the AUC will range from 100 to 400. PS and PA will be different compared to their respective baselines. These values will depend upon the actual anti-platelet medication that the patient is taking. The anti-platelet medication sensitive and the anti-platelet medication non-responders will show differences from baselines; sensitive individuals will show less aggregation. In certain embodiments, an algorithm that combines and categorizes this data, into an actionable form useful to the physician may be employed.

In another embodiment, no baseline readout is obtained. In this situation, it may be desirable, but is not necessary, to use a previous test run for this individual before the individual started the anti-platelet medication therapy as the baseline readout. In the situation where no baseline is obtained, the assay involves performing one or more platelet aggregation assays whereby a first platelet rich sample or whole blood sample is obtained from an individual and is combined with synthetic collagen to form a treated sample. The sample is measured to obtain a readout to determine the individual's level of platelet aggregation. In certain embodiments, an initial platelet aggregation assay may be performed to check for spontaneous aggregation. The results of this treated sample are used to determine the individual's anti-platelet medication response status. For example, if the individual shows a significant reduction in platelet aggregation, then the individual may be characterized as normal or having an average anti-platelet medication sensitivity. If the individual shows very little inhibition of platelet aggregation after taking the anti-platelet medication (i.e. the platelets still aggregated after the individual ingested the anti-platelet medication), then the individual may be characterized as being non-responsive to the anti-platelet medication (thrombosis or clotting risk). If the individual showed an almost complete lack of platelet aggregation after ingesting the anti-platelet medication, then the individual may be characterized as being hypersensitive to the anti-platelet medication (bleeding risk). Either is deadly.

In the case where the platelet aggregation utilizes LTAAs, the readout may be area under the curve, slope, primary aggregation, lag phase (LP), disaggregation (DA), final aggregation (FA), or a combination thereof. Additional analysis methods may be employed such as obtained with a NARX mathematical analysis or other computational models. Norms and/or established cut off values for various different readouts (i.e. PA, PS or AUC) for each separate medication may be obtained by performing a representative number of assays on different patient groups to determine cut off values for categorizing an individual as hypersensitive, non-responder or normal. Thus, in assays of the present invention, the determination of an individual's platelet sensitivity status may involve comparison against the own individual's baseline or previous LTAA result and/or may involve a comparison against established values. Further, dilution profile used in the present invention may also be visually analyzed in a manner similar to that of reading an EKG.

### A2. Testing for Platelet Sensitivity to Anti-Platelet Medication in an Individual (Post Medication Readouts—No Baseline)

In certain situations, such as in the emergency clinical setting when it is not feasible to obtain a baseline (pre-anti-platelet medication baseline) or when one cannot determine from the patient whether he or she has been on anti-platelet medication therapy or has been compliant in taking their anti-platelet medication, it may be desirable to have a test that tests for the individual's platelet sensitivity. For example, a patient may be suspected of being on an anti-platelet medication but that fact may not be verifiable because of the patient's condition, one could use embodiments of the invention to test for platelet activity before proceeding onto other medical procedures that might involve risks of bleeding.

The final in-test concentration of synthetic collagen used for this embodiment and other embodiments described in the application ranges from about 2 ng/mL to about 500 ng/mL; from about 8 ng/mL to about 500 ng/mL; from about 10 ng/mL to about 500 ng/mL; from about 15 ng/mL to about 500 ng/mL; from about 20 ng/mL; from about 25 ng/mL to about 500 ng/mL; from about 30 ng/mL to about 300 ng/mL; from 40 ng/mL to about 400 ng/mL; from about 50 ng/mL to about 500 ng/mL; from about 8 ng/mL to about 100 ng/mL; from about 8 ng/mL to about 80 ng/mL; from about 8 ng/mL to about 50 ng/mL. Preferably the amount of synthetic collagen is at least about 8 ng/mL or at least about 2 ng/mL. These values and ranges are preferably used when the platelet aggregation tests are light transmission assays and the sample is a PRP sample that is tested. However, they may also be used in other analyzers, including flow cytometers and impedance aggregometers or their equivalents.

In certain embodiments, an initial platelet aggregation assay may be performed to check for spontaneous aggregation to test for whether the platelets have any inherent hyperactivity. Saline is added to the LTAA instead of the synthetic collagen to see if there is any aggregation.

In certain embodiments, preferably a platelet rich sample is obtained from the patient and is treated with synthetic collagen. The sample is then analyzed to obtain a readout. The readout may be analyzed to determine the patient's platelet reactivity status and the potential risk for blood clotting complications or bleeding complications. The analysis may involve a comparison against readouts previously characterized as being a normal, hyper-responders or non-responders. For example in a normal/healthy platelet aggregation response (e.g. there is a healthy amount of inhibition of platelet aggregation in the presence of synthetic collagen); a non-responder and hence a clotting risk (there is a complete or quick and long lasting aggregation response—e.g. no or very little inhibition of platelet aggregation) or a hypersensitive responder and hence a bleeding risk (there is little inhibition of platelet aggregation in the presence of synthetic collagen as compared to a normal response).

In the case of using LTAAs to measure platelet aggregation (or inhibition of platelet aggregation), the readout from the LTAA may be preferably the area under the curve, slope, primary aggregation, lag phase (LP), disaggregation (DA), final aggregation (FA), or a combination thereof. In certain embodiments the preferred readout is area under the curve.

For example, when using the Bio/Data's PAP 8E aggregometer, and when using an “in-test” concentration of 50 ng/mL of synthetic collagen, for an anti-platelet medication hypersensitive individual, the baseline for PA will range from 50% to 100%. The baseline for PS will range from 25 to 60. The baseline for AUC will range from 300 to 800. After the anti-platelet medication, the AUC will range from 100 to 400. PS and PA will be different compared to their respective baselines. These values will depend upon the actual anti-platelet medication that the patient is taking. The anti-platelet medication sensitive and the anti-platelet medication non-responders will show differences from baselines; sensitive individuals will show less aggregation. In certain embodiments, an algorithm that combines and categorizes this data, into an actionable form useful to the physician may be employed.

### B. Testing for Platelet Sensitivity to an Anti-Platelet Medication in an Individual Using a Dilution Profile

B1. Dilution Profile Before and after Ingestion of Anti-Platelet Medication

In another embodiment, a dilution profile of synthetic collagen is utilized across a number of different platelet aggregation assays such as LTAAs run on an individual's PRP or whole blood sample. In this embodiment of the invention, more than two reactions (more than just the baseline test before the anti-platelet medication ingestion and the test after the anti-platelet medication ingestion) are run. A series of platelet aggregation assays are run using multiple differing amounts of synthetic collagen. This is referred to herein as the “dilution profile platelet aggregation assays” or “dilution profile LTAAs” or “dilution profiles.” In this embodiment, multiple different PRP or whole blood samples are obtained from the individual before the anti-platelet medication ingestion (to obtain a baseline dilution profile) and after the anti-platelet medication ingestion (to obtain a post anti-platelet medication dilution profile). Each individual pre-anti-platelet medication platelet sample is mixed with a different amount of synthetic collagen (ranging from 2 ng/mL to 500 ng/mL) and a platelet aggregation assay is performed on each sample to obtain a baseline dilution profile over the range of concentrations. Then the individual is given the anti-platelet medication and sufficient time is allowed to pass to ensure the anti-platelet medication has been metabolized. Then, multiple PRP or whole blood samples are obtained from the individual post anti-platelet medication ingestion and mixed with different amounts of synthetic collagen. Platelet aggregation assays are performed on each sample to obtain a post-anti-platelet medication dilution profile. The same concentrations of synthetic collagen that were used in the pre-anti-platelet medication baseline platelet aggregation assays are preferably used in the post-anti-platelet medication platelet aggregation assays. The results are analyzed and the changes in platelet aggregation between the pre- and post-anti-platelet medication assays, as well as changes occurring over the different amounts of synthetic collagen in the dilution profile are studied to determine the individual's anti-platelet medication sensitivity response (whether the individual is anti-platelet medication hypersensitive, average anti-platelet medication sensitive or anti-platelet medication non-responsive and the degree of sensitivity therein). In certain embodiments, the change in PA, PS, AUC, LP, DA, FA or a combination thereof between the pre- and post-anti-platelet medication LTAAs, as well as changes in the PA, PS or AUC or a combination thereof over the different amounts of synthetic collagen, are studied (often using an algorithm that categorizes the data or information and reports the data) to determine the individual's anti-platelet medication sensitivity response (whether the individual is anti-platelet medication hypersensitive, average anti-platelet medication sensitive or anti-platelet medication non-responsive and the degree of sensitivity therein). In certain embodiments, the results are characterized using the aggregometer's proprietary algorithm embedded in system software, which makes the analysis easier for the diagnostician to understand the test results and therefore make appropriate clinical decisions.

The final in-test concentration of synthetic collagen used for this embodiment and other embodiments described in the application ranges from about 2 ng/mL to about 500 ng/mL; from about 8 ng/mL to about 500 ng/mL; from about 10 ng/mL to about 500 ng/mL; from about 15 ng/mL to about 500 ng/mL; from about 20 ng/mL; from about 25 ng/mL to about 500 ng/mL; from about 30 ng/mL to about 300 ng/mL; from 40 ng/mL to about 400 ng/mL; from about 50 ng/mL to about 500 ng/mL; from about 8 ng/mL to about 100 ng/mL; from about 8 ng/mL to about 80 ng/mL; from about 8 ng/mL to about 50 ng/mL. Preferably the amount of synthetic collagen is at least about 8 ng/mL or at least about 2.0 ng/mL. These values and ranges are preferably used when the platelet aggregation tests are light transmission assays and the sample is a PRP sample that is tested. However, they may also be used in other analyzers, including flow cytometers and impedance aggregometers or their equivalents.

B2. Dilution Profile Only after Ingestion of Anti-Platelet Medication (Baseline does not Involve Dilution Profile)

In other embodiments, the pre-anti-platelet medication baseline is established with one platelet aggregation assay performed using one concentration of synthetic collagen (such as, but not limited to, 50 ng/mL) on an individual pre-anti-platelet medication platelet sample, whereas multiple different concentrations of synthetic collagen (such as, but not limited to, 2.0 ng/mL; 8 ng/mL, 12 ng/mL, 25 ng/mL; 32 ng/mL, 50 ng/mL, 100 ng/mL, 250 ng/mL and/or 500 ng/mL) are still used in different post anti-platelet medication platelet aggregation assays to create the post-anti-platelet medication dilution profile. In this case, the results are analyzed and the change in platelet aggregation from differing amounts of synthetic collagen are studied, and compared against each other as well as against the baseline (pre-anti-platelet medication) platelet aggregation to determine the donor's anti-platelet medication sensitivity response (whether anti-platelet medication hypersensitive, normal/average anti-platelet medication sensitive or anti-platelet medication non-responsive and the degree of sensitivity therein). In the case where the platelet aggregation assay is LTAAs the PA, PS, AUC, LP, DA, FA or a combination thereof is used as the readout to measure and compare platelet aggregation test results.

In other embodiments, a pre-anti-platelet medication baseline or pre-anti-platelet medication dilution profile is not obtained. This may be useful in the emergency clinical setting when it is not feasible to obtain a pre-anti-platelet medication baseline or whether one cannot determine from the patient whether he or she has been on anti-platelet medication therapy. In this embodiment, multiple different platelet rich plasma or whole blood samples are obtained from the individual and each are mixed independently with a different synthetic collagen concentration to obtain multiple different treated samples for the dilution profile assays. Platelet aggregation assays are performed for each of these samples to obtain a dilution profile over the range of different concentrations of synthetic collagen. The data is obtained and measured. For example, when the platelet aggregation assay uses LTAAs, the AUC, PA, PS, LP, DA, FA or a combination therefore are obtained and analyzed over the different ranges of synthetic collagen. In certain embodiments the results are analyzed using the aggregometer's proprietary algorithm embedded in system software.

The inventors have determined that this embodiment can be used to predict the individual's platelet the anti-platelet medication response. For individuals having an average or “normal” anti-platelet medication sensitivity, when looking at the slope, percentage aggregation and/or the AUC, over the dilution profile, the slope, percentage aggregation and/or the AUC will show a corresponding decrease along with the decrease in the amount of synthetic collagen used. Thus, for example, within a given range of various dilutions of synthetic collagen, as the concentration goes down, so will the slope, percentage aggregation, and the AUC. There seems to be an almost linear decrease in slope, percentage aggregation and AUC that runs almost parallel or has almost a direct correlation with the concentration of synthetic collagen. On the other hand, for individuals who are anti-platelet medication non-responders, instead of having a slope, percentage aggregation, and/or the AUC that has a linear-like decrease that corresponds with the decrease in synthetic collagen, there is a point along the dilution profile where there is an increase in slope, an unexpected temporary increase in percentage aggregation and/or a temporary increase in AUC when there should be a decrease (because the concentration of synthetic collagen decreases). Then, as the dilution profile continues to decrease, the slope and AUC “bounce back” down to where it should be (based on the dilution of synthetic collagen) and where it was before the temporary increase and then continues along decreasing. The point at which the bounce back will occur will be at different concentrations of synthetic collagen, and will depend upon the medication class and dose, as well as the patient's metabolic and platelet receptor genetics.

Anti-platelet medication hypersensitive individuals will show increases in PA, PS and AUC compared to expected/normal results, sometimes called the reference range.

In certain embodiments of the invention utilizing the dilution profile concept, a series of 7, 6 or 5 different concentrations are used to develop a dilution profile, and in other embodiments, 4 different concentrations are used and yet in other embodiments, 3 or 2 different concentrations are used. Using too many different concentrations can make the test cumbersome and time consuming, whereas using too few concentrations reduces the amount of data obtained and limits the sensitivity analysis. It is likely that the use of many different concentrations will be useful in personalizing a patient's drug therapy, whereas for rapid or emergency screening of a patient or immediately prior to procedures such as a cardiac catheterization or open heart surgery, it is likely that just one or two different platelet aggregation assays would be run. Test kits may have prefilled vials of diluent to preclude dilution errors in the field and further simplify performing the test.

The range of synthetic collagen used is the dilution profile is preferably within the “anti-platelet medication sensitive range,” which is defined herein as the range of concentrations in which in an average anti-platelet medication sensitive individual the measured platelet activity/aggregation is reduced corresponding with decreasing amounts of synthetic collagen concentrations (e.g. the AUC and/or the slope decreases with the concentration of collagen).

In certain embodiments the different synthetic collagen dilution amounts comprise multiple different synthetic collagen amounts chosen from within the concentration range from about 2 ng/mL to about 500 ng/mL; from about 8 ng/mL to about 500 ng/mL; from about 10 ng/mL to about 500 ng/mL; from about 15 ng/mL to about 500 ng/mL; from about 20 ng/mL; from about 25 ng/mL to about 500 ng/mL; from about 30 ng/mL to about 300 ng/mL; from 40 ng/mL to about 400 ng/mL; from about 50 ng/mL to about 500 ng/mL; from about 8 ng/mL to about 100 ng/mL; from about 8 ng/mL to about 80 ng/mL; from about 8 ng/mL to about 50 ng/mL. These values and ranges are preferably used when the platelet aggregation tests are light transmission assays and the sample is a PRP sample that is tested. However, they may also be used in other analyzers, including flow cytometers and impedance aggregometers or their equivalents.

For example, in certain embodiment there are seven different synthetic collagen amounts as the final “in test” concentration as follows: 500 ng/mL; 325 ng/mL; 250 ng/mL; 150 ng/mL; 100 ng/mL; 75 ng/mL; and 50 ng/mL. In other embodiments, there are different synthetic collagen amounts (2, 3, 4, 5, 6 or 7 different dilutions) ranging from 8 ng/mL to 250 ng/mL to about. Any number chosen from about 8 ng/mL to about 500 ng/mL can be used for the different amounts chose for the dilution profile concentrations. For example in one 7 dilution profiles the amounts chosen are 25, 50, 75, 100, 125, 150 and 175 ng/mL. In another, non-limiting example, the 7 dilution profiles chosen are 25, 50, 100, 150, 200, 250 and 300 ng/mL. As another non-limiting example, the 7 dilution profiles chosen are 50, 100, 150, 225, 300, 350 and 500 ng/mL. The idea is that any number can be chosen from 25 to 500 and preferably in the profiles the different concentrations are spread out across that range. It is the same for when 2, 3, 4, 5 or 6 different dilutions are used. Because it would be too cumbersome to list every possible dilution that could be used, when a range is listed herein, it means to include every number that falls within that range including the first and last number in that range.

### C. Testing for Patient Compliance

In another embodiment of the invention, there is provided a method of testing/monitoring patient compliance in taking the prescribed doses of anti-platelet medication and residual platelet activity using assays discussed above with synthetic collagen. Non-compliance includes not taking the medication, not taking the proper dose or not staying with the effective dosing (time) window. Recent studies have shown that a large problem in health care is patient noncompliance. Current thinking is that what was once thought to be drug resistance may instead be a manifestation of non-compliance complicated by the use of multiple, non-standardized laboratory tests to evaluate platelets inhibited response to the anti-platelet medication.

Accordingly, to measure compliance the patient can be routinely tested, such as once a week, bi-monthly, monthly, every three months, etc., and the results compared against each other. If the aggregation results vary widely from one test to another, the patient can be further tested to determine if anti-platelet resistance (resistance to the anti-platelet medication) has developed or the patient could be questioned as to his compliance in taking the prescribed doses of the medication and following the dosing schedule. The aggregation tests may reveal variability from test to test and this variability could be used as an indicator that the patient has not been following the prescribed regular dosing regimen (either not taking the dose every day or taking the dose at different times of the day). Further testing could be performed to determine if the patient should be on a dual therapy of aspirin and the anti-platelet medication or perhaps a regimen of a different anti-platelet medication.

Methods involve obtaining a PRP or whole blood sample from the patient and testing for platelet aggregation in the presence of synthetic collagen in assays as described above. The results of the platelet aggregation assays are compared against prior results obtained for that patient. If the results are similar to previous results, it may be determined that the patient has been compliant in the therapy. If the results are different or vary substantially, then compliance or development of resistance to the medication may be an issue. For example, if the results before showed that the patient had a certain level of platelet aggregation and the later tests showed that there was more platelet aggregation then it could be that the patient had not been taking the anti-platelet medication or developed resistance.

It is preferable that the same level of synthetic collagen is used in the compliance testing over time so the comparisons against previous tests are valid. In addition, it is preferable for the same reasons that the same method of measuring platelet aggregation (e.g. LTAAs) and same readout (e.g. area under the curve) are compared. There will likely be slight variations between the patient's test results over time but significant differences between the tests are to be considered as likely indicators of noncompliance or resistance (i.e. the tests showed substantially more platelet aggregation than previous tests). In some cases, the patient might have started taking aspirin on his own volition and if the tests results showed less platelet aggregation as compared to previous tests, the patient may be questioned about the aspirin and counseled against such course of action if the physician believed a bleeding risk might be present or increased.

The final in-test concentration of synthetic collagen used for this embodiment and other embodiments described in the application ranges from about 2 ng/mL to about 500 ng/mL; from about 8 ng/mL to about 500 ng/mL; from about 10 ng/mL to about 500 ng/mL; from about 15 ng/mL to about 500 ng/mL; from about 20 ng/mL; from about 25 ng/mL to about 500 ng/mL; from about 30 ng/mL to about 300 ng/mL; from 40 ng/mL to about 400 ng/mL; from about 50 ng/mL to about 500 ng/mL; from about 8 ng/mL to about 100 ng/mL; from about 8 ng/mL to about 80 ng/mL; from about 8 ng/mL to about 50 ng/mL. Preferably the amount of synthetic collagen is at least about 8 ng/mL or at least 2 ng/mL. These values and ranges are preferably used when the platelet aggregation tests are light transmission assays and the sample is a PRP sample that is tested. However, they may also be used in other analyzers, including flow cytometers and impedance aggregometers or their equivalents.

### D. Predicting Effectiveness of an Anti-Platelet Medication

Further, the present invention also provides assays that can test the effect of an anti-platelet medication on the platelet activity and thus assist a physician in predicting the effectiveness of that anti-platelet medication for a certain or individual patient. See example 2. Thus, if a physician was considering starting a patient on an anti-platelet medication, the patient's PRP or whole blood sample could be taken and treated with an anti-platelet medication. After adding synthetic collagen, platelet aggregation is studied. If it is determined that the level of platelet aggregation is acceptable, then the physician may prescribe that medication. Or if the level of platelet aggregation was not acceptable, the physician may test and prescribe a different anti-platelet medication or a combination of medications. In certain embodiments, the physician may compare the aggregations results against results of previous patients where the same anti-platelet medication was prescribed and was determined to be beneficial to assist in the prediction of whether the anti-platelet medication would be beneficial for this particular patient.

In these embodiments the final in-test concentration of synthetic collagen that is used tests the ability of the platelets to aggregate in the presence of an agonist (synthetic collagen). The final in-test concentration of synthetic collagen used for this embodiment and other embodiments described in the application ranges from about 2 ng/mL to about 500 ng/mL; 8 ng/mL to about 500 ng/mL; from about 10 ng/mL to about 500 ng/mL; from about 15 ng/mL to about 500 ng/mL; from about 20 ng/mL; from about 25 ng/mL to about 500 ng/mL; from about 30 ng/mL to about 300 ng/mL; from 40 ng/mL to about 400 ng/mL; from about 50 ng/mL to about 500 ng/mL; from about 8 ng/mL to about 100 ng/mL; from about 8 ng/mL to about 80 ng/mL; from about 8 ng/mL to about 50 ng/mL. Preferably the amount of synthetic collagen is at least about 8 ng/mL, or is at least 2.0 ng/mL. These values and ranges are preferably used when the platelet aggregation tests are light transmission assays and the sample is a PRP sample that is tested. However, they may also be used in other analyzers, including flow cytometers and impedance aggregometers or their equivalents.

The concentration of medicine that is used in the test depends on the medication and the concentration of the synthetic collagen. Because synthetic collagen induced platelet aggregation is a functional test, there is no need to have genetic and metabolic test data available prior to prescribing the individualized anti-platelet therapy. The concentration of medicine that is used may depend on the dosing that is given to a patient and the desired levels obtained in the plasma. To determine the best concentrations to use in the method of the invention, one skilled in the art could follow example 2 and obtain healthy donors and test varying ranges of the anti-platelet medication and the synthetic collagen, using the plasma levels as a guide to determine the best ranges (the most sensitive and most reliable across a collection of healthy donors) of anti-platelet medication and synthetic collagen.

As seen above concerning the various embodiments, it is apparent that varying amounts of synthetic collagen can be used, depending upon the test to be performed or the clinician's request for an assessment. The amount of synthetic collagen used is about 100 fold less than what is generally used when performing LTAAs with biological source collagen. For example, usually LTAAs using calf skin biological collagen generally use 0.19 mg/mL (milligrams/mL) collagen (as the “in-test” concentration); and LTAAs using equine tendon collagen generally use 2.0 μg/mL (micrograms/mL) collagen in the LTAA test (as the “in-test” concentration), whereas generally the methods of the present invention utilize from about 500 ng/mL to about 25 ng/mL (nanogram/mL) of synthetic collagen in each LTAA test (as the “in-test” concentration).

When testing platelet aggregation using flow cytometry, the usual concentrations of biological collagen ranges from 0.01-100 μg/mL, with 20 μg/mL to be most common. However, using synthetic collagen, the amounts used are much lower, ranging from about 2.0 ng/mL to about 640 ng/mL.

In certain embodiments, when the aggregation assay uses flow cytometry to measure aggregation, the amount of synthetic collagen used as in the in-test collagen ranges from 2 ng/mL to 64 ng/mL. In certain embodiments, the amounts of in-test synthetic collagen ranges from 4 ng/mL to 64 ng/mL; from 6 to 64 ng/mL; from 8 ng/mL to 64 ng/mL; from 2 ng/mL to 100 ng/mL; from 4 ng/mL to 100 ng/mL; from 6 to 100 ng/mL; from 8 to 100 ng/mL; and any subset of ranges or individual numbers from 2 ng/mL to 100 ng/mL. In certain embodiments the amounts of in test synthetic collagen is 2 ng/mL, 4 ng/mL, 6 ng/mL, 8 ng/mL, 16 ng/mL, 32, ng/mL and/or 64 ng/mL. In certain embodiments the amount of in-test synthetic collagen is any number in the range of 2 ng/mL to 100 ng/mL, such as, but not limited to 2 ng/mL, 3, 4, 5, 6, 7, 8 . . . 95, 96, 97, 98, 99, or 100 ng/mL.

Although there are ranges included hereinabove, the present invention is not limited by the recitation of the first and last endpoint to only mean the first and last, but expressly includes the first and last endpoint as well as all of the concentrations within the endpoints. It would be just too cumbersome herein to list every concentration that falls within the recited ranges. The inventors have contemplated using more than one concentration, and more than one range as well as more than one concentration within the recited range.

The ability to use such low concentrations as well as dilution profiles of collagen is only available with the synthetic collagen. When the inventors tried to dilute biological collagen down to similar low concentrations, it became physically impossible to dilute down the biological collagen to the levels anywhere close to that used in the LTAAs of the present invention with synthetic collagen. Biological collagen is an insoluble, viscous, heterogeneous material, and has long structured fibrous proteins wound into a triple helix. These physical properties precluded the ability to dilute the biological collagen to any low concentration even 100 fold close to the synthetic collagen or to have any such dilution perform in a predictable or useful manner. Further the LTAAs did not work (no aggregation occurred) when using calf skin collagen at a concentration a little lower than 0.19 mg/mL (milligrams/mL) (as the “in-test” concentration); nor when using equine tendon collagen a little lower than 2.0 μg/mL (micrograms/mL).

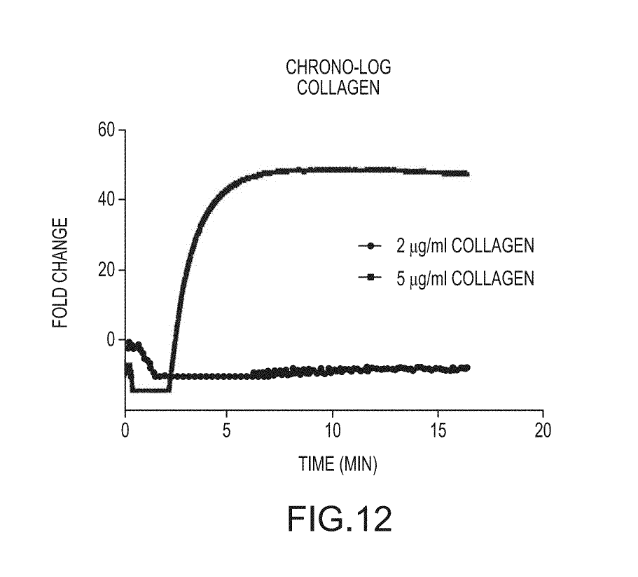

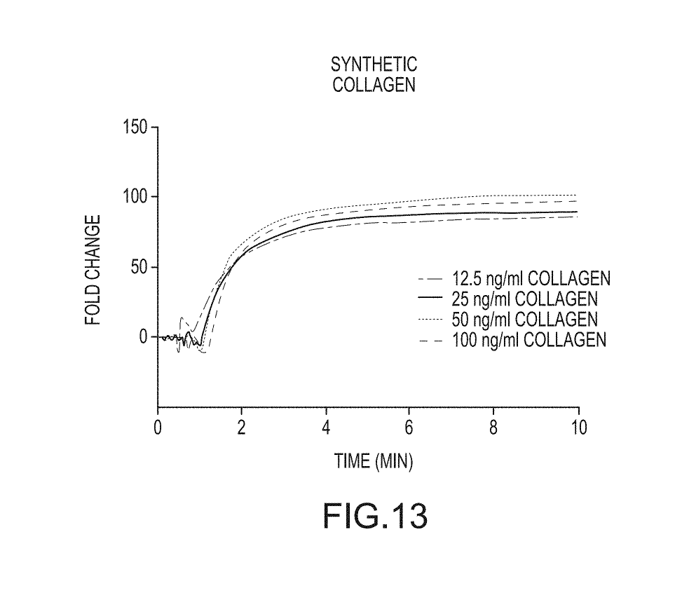

In tests performed on whole blood, it was shown that diluting biological collagen did not produce any viable results but synthetic collagen could be diluted from 100 ng/mL to 12.5 ng/mL and still elicit the same response. See FIGS. 12 and 13.

Although there are ranges included hereinabove, the present invention is not limited by the recitation of the first and last endpoint to only mean the first and last, but expressly includes the first and last endpoint as well as all of the concentrations within the endpoints. It would be just too cumbersome herein to list every concentration that falls within the recited ranges. The inventors have contemplated using more than one concentration, and more than one range as well as more than one concentration within the recited range.

In preferred embodiments the LTAAs will use dilution profiles. The results (profiled data) of the LTAAs will be compared to some known expected profile. One type of comparison that will be used is “cumulative reports” (CR). A CR is a serial presentation of a patient's test results over time, and is particularly useful in identifying changes in a patient's response over time. So, the expected results used for comparative purposes are the patient's own prior test result(s). In other embodiments, an “expected profile” or “normal profile” is compared against the patient's profile. An “expected profile” or “normal profile” for each drug and possible for each ethnic group will likely be developed and used as a comparison basis.

### Synthetic Collagen

In certain embodiments the synthetic collagen is described in U.S. patent application Ser. No. 12/520,508, which is herein incorporated by reference in its entirety. In certain embodiments, the synthetic collagen is a synthetic collagen that has the ability to self-assemble into a triple helix to form fibrils, which allows the synthetic collagen to mimic type I collagen. In certain embodiments the synthetic collagen comprises a polypeptide having a peptide fragment represented by the formula (I)

-(Pro-X-Gly)ₙ (I)

wherein X represents Hyp; and n represents an integer of from 20 to 5,000; and wherein the polypeptide has a molecular weight at a range of from 10,000 to 500,000,000. In certain embodiments, the synthetic collagen having the structure of formula (I) has the ability to self-assemble into a triple helix to form fibrils, which allows the synthetic collagen to mimic type I collagen. It is preferred that synthetic collagen used in all the assays of the present invention have the ability to self-assemble into a triple helix to form fibrils, which allows the synthetic collagen to mimic human type I collagen.

In certain embodiments, the synthetic collagen that is used is described in U.S. Pat. No. 7,262,275. The synthetic collagen molecule was made by the method described in U.S. Pat. No. 7,262,275 (See e.g. Example 6 and Example 7). The molecular weight of the molecule was measured by the method described in the example section in the same patent as was over 1,000,000.

In certain embodiments the synthetic collagen has the following values based on GPC-MALs (gel permeation chromatography—multi-angle laser light scattering); Average molecular weight (Mₙ) 1.3×10⁴; M_{w} (weight average molecular weight)=1.6×10⁴; size average molecular weight (M_{z}) 2.0×10⁴. In other embodiments, the synthetic collagen as has the following values based on GPC-MALs (gel permeation chromatography—multi-angle laser light scattering); Average molecular weight (Mₙ) 2.8×10⁴; M_{w} (weight average molecular weight)=4.1×10⁴; size average molecular weight (M_{z}) 6.1×10⁴.

The synthetic collagen can be measured by GPC-Mals. The synthetic collagen molecules tested in the present invention were measured using the HLC-8120GPC device manufactured by Tosoh with the following conditions.
Column: TSKgel α-M (7.8 mm I.D.×30 cm)×2 (manufactured by Tosoh).
Density Detector: Differential refractometer (RI detector), polarity=(+).
MALS: DAWN HELEOS (manufactured by Wyatt Technology).
MALS Laser wave: 658 nm.
Eluent: HFIP (1,1,1,3,3,3-Hexfloro-2-propanol) manufactured by central glass+5 mM-CF₃COONa (1^{st} class manufactured by Wako Pure Chemical).
Flow Speed: 0.6 mL/min.
Column Temp.: 40° C.
RI detector Temp.: 40° C.
MALS Temp.: Room Temp.
Sample density: 2 mg/mL.
Sample amount: 100 μL.
Pre-treatment of sample: After weighing the samples, they were dissolved by adding a given amount of eluent and left at room temperature overnight. The samples gently mixed and then were then filtered through a 0.5 μm PTFE cartridge filter.

In certain embodiments n is an integer of 20 to 250. In certain embodiments n is an integer of 20 to 200. In certain embodiments n is an integer of 20 to 150. In certain embodiments n is an integer of 30 to 100. In certain embodiments n is an integer of 20 to 2,500; of 20 to 2,000; of 20 to 1,500; of 20 to 1,000; of 20 to 500; or of 20 to 250; 30 to 2,500; of 30 to 2,000; of 30 to 1,500; of 30 to 1,000; of 30 to 500; or of 30 to 250. It is preferred that the synthetic collagen molecules discussed above have the ability to self-assemble into a triple helix to form fibrils, which allows the synthetic collagen to mimic type I collagen.

Two factors to consider in choosing the synthetic collagen is solubility and ease of handling. If the molecular weight is too small, the synthetic collagen may have poor solubility characteristics. If the molecular weight is too large, the synthetic collagen may not have good handling characteristics (may be too viscous and may have poor dispersibility). Thus, a preferred synthetic collagen of the formula (I) [-(Pro-X-Gly)ₙ] has both good solubility and good handling characteristics.

The following synthetic collagen molecules were tested: n=24 (Mn=6,300); n=28 (Mw=7,500); n=49 (Mn=13,000); n=60 (Mw=16,000); n=75 (Mz=20,000); n=105 (Mn=28,000); n=153 (Mw=41,000); n=229 (Mz=61,000). When testing various synthetic molecules, those having the n value from between 49-75 showed the best combination of desirable solubility and handling characteristics.

In certain embodiments of the invention, the synthetic collagen may be all one length (for example where n=49 for all synthetic molecules and in certain embodiments, the synthetic collagen may be a mixture of many different lengths (for example, but not limited to, the synthetic collagen is a mixture of molecule having n from 49-75).

### Kits

The present invention also provides kits useful for testing platelet aggregation in a light transmission assay, comprising a synthetic collagen. The synthetic collagen is as described above and can be at many different concentrations. In addition, the kit may comprise one or more diluents as well as controls.

The synthetic collagen can be supplied at a higher concentration in the vial than what would be used as the “in-test” concentration. In certain embodiments, the synthetic collagen in the vial is preferably more than 10 times the amount of the final “in-test” concentration desired.

In certain embodiments the synthetic collagen is supplied in the kit at the concentration contemplated for use in the methods of the present invention to bypass the need to create dilutions of the synthetic collagen. In other words, the synthetic collagen is provided so that it is in the concentration that would be used directly in the methods of the present inventions.

In other embodiments the vial could contain a higher concentration amount and the directions included in the kit would provide instructions on the desired concentration to use in the assay to achieve the desired final “in-test” concentration of synthetic collagen.

In other embodiments, the kit contains at least one single use vial and/or at least one multiple use vial of synthetic collagen. For a single use vial, the vial would contain only the amount of synthetic collagen needed for one LTAA. For a multiple use vial, the synthetic collagen may be supplied at the desired in-test concentration, but the vial contains more than the amount of volume needed for more than one LTAA.

The kits of the present invention preferably contain instructions for use of the synthetic collagen in the light transmission assay using methods described herein.

In other embodiments, kits of the present invention contain more than one vial of synthetic collagen at the same concentration or in other embodiments the kits contain more than one vial at a different concentration. Kits having more than one vial at different concentrations would be useful in the dilution profile LTAAs of the present invention. For example, one kit of the present invention may contain vials having 7, 6, 5, 4, or 3 different concentrations of synthetic collagen ranging. Each vial would, in certain embodiments, provide the synthetic collagen at the desired final “in-test” concentration and could be supplied as a single use or a multiple use vial.

The present invention is based on measuring platelet aggregation capacity along with dilution profiles generated (in certain embodiments) using synthetic collagen and subsequent data analysis and actionable report. Other methods for measuring platelet response and other measurements to one or more anti-platelet therapy regimens using the synthetic collagen are also expected to be useful, where currently these methods are reported to be of limited clinical value. For example, the whole blood and point of care platelet function analyzers, methods, and technologies such as impedance and multiple electrode impedance aggregometry, high shear stress, cone and plate, flow cytometry, and other-point—of care technologies and assays of platelet function or inhibition.

It has been discovered that the nature of the vial used to store biologic or synthetic collagen can affect the collagen by activating the collagen to some degree. It is preferable that the container used to store the synthetic collagen does not activate the collagen to ensure that when the synthetic collagen is removed from the vial and is introduced into a test system, the degree of activation and adherence of the synthetic collagen is due only to that test system. In other words, artifacts caused by unintentional activation by the interaction of the collagen with the container are not introduced into the aggregation assays. Collagens, including synthetic collagen, stored in generic polypropylene vials or containers are activated to an unknown degree, subsequently adhere to the container, and are thus not available to participate in the test system. The amount of collagen unavailable to the test system because it has adhered to the container and/or cap is unknown and, based on stability data, is variable. The inventors have discovered that the use of synthetic collagen that has been prepared and stored in a homopolymeric container eliminates a significant degree of variability in test results. Accordingly, it is preferred that the synthetic collagen is prepared and stored in a homopolymeric container.

Most containers that are noted as polypropylene are not a single plastic but rather are a family of plastics whose performance can be modified by including various additives during the manufacturing process. Thus, the manufacturing process itself could produce different variations of polypropylene. Further, the nature of the additives is largely unknown or disclosed to the purchaser/public as this information is considered proprietary by the manufacturers. In addition, mold release agents add another variable that could not be assessed.

It was discovered that containers that have the best long term stability and do not interact with the synthetic collagen have the following characteristics: a) the chemical structure is based on a specific, identical monomer that is repeated (a homopolymer—a polypropylene polymer consisting of identical monomer units); b) caps are made of the same material as the tubes; and c) the caps have an additional internal seal such as a silicone O ring or washer or have a secondary seal molded therein. Exemplary vials include cryovials and caps obtained from Simport (T310 Series); Lake Charles Manufacturing (54A series), and BD Falcon tubes 352096 series).

In addition, the inventors discovered that better stability was achieved when the synthetic collagen was diluted with physiologic saline (with or without Thimerosal as a preservative) instead of purified water. Accordingly, kits of the present invention may contain vials of saline for dilution of synthetic collagen.

### EXAMPLES

### Example 1: Evaluation of Synthetic Collagen Using Flow Cytometry

### Materials:

1. Collagen soluble calf skin; Bio/Data

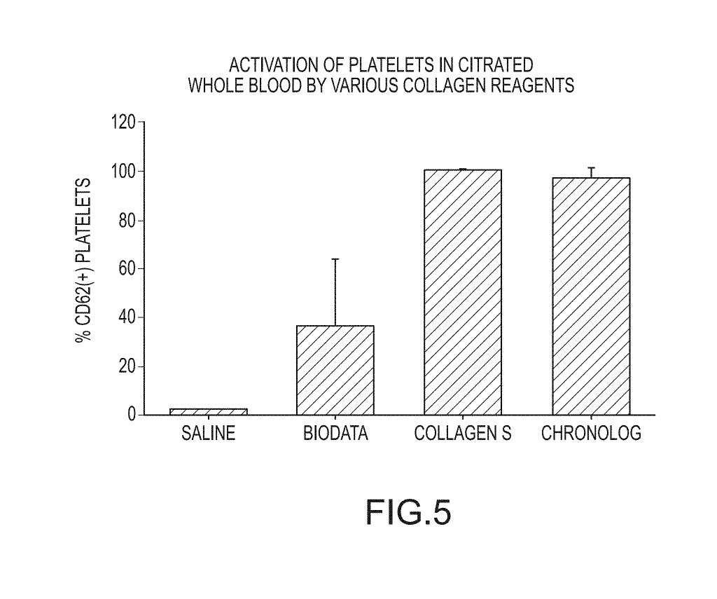

2. Synthetic Collagen (referred to in FIGS. 5-8 as Collagen S)

3. Collagen—type I equine; Chrono Log

4. ReoPro—2.5, 5, 10 μg/mL final concentrations

5. Integrilin—1, 2, 5 μg/mL final concentrations

6. Aggrastat—1, 2, 5 μg/mL final concentrations

### Methods: Flow Cytometry

A vial of Bio/Data calf skin collagen was reconstituted with 0.5 mL of water to make a 1.9 mg/mL solution, A vial of Synthetic collagen was reconstituted with 1 ml of Synthetic collagen diluent to make a 0.0005 mg/mL solution. Chrono Log collagen was diluted with saline to make a 100 μg/mL solution. A stock 2% paraformaldehyde solution was diluted with calcium-free Tyrode's buffer to make a 1% paraformaldehyde solution. A set of tubes containing 1 mL of 1% paraformaldehyde was prepared. A second set of tubes which contained 30 μl of collagen reagent and 30 μl of anti-platelet drug was prepared and set in a 37° C. heating block. Whole blood was drawn from healthy individuals into sodium citrate. 240 μl of citrated blood was added to the tubes at 15-20 second intervals and gently mixed. After a 3 minute incubation period, 50 μl of activated blood was transferred to the corresponding paraformaldehyde-containing tube. After a 30 minute incubation at 4° C., the samples were centrifuged at 1,600 rpm for 10 minutes and the supernatant was removed. The cell pellet was resuspended in 750 μl of Tyrode's buffer. 10 μl each of CD61FITC and CD62PE (BD Biosciences) was added to a set of clean tubes. 100 μl of resuspended cells was added to the antibody tubes. After a 30 minute incubation period in the dark at room temperature, 700 μl of Tyrode's buffer was added to each tube and the samples were analyzed on the flow cytometer (EPICS-XL, Beckman-Coulter). Platelet activation was assessed in terms of the percentage of platelets expressing P-selectin and the percentage of aggregated platelets.

### Results:

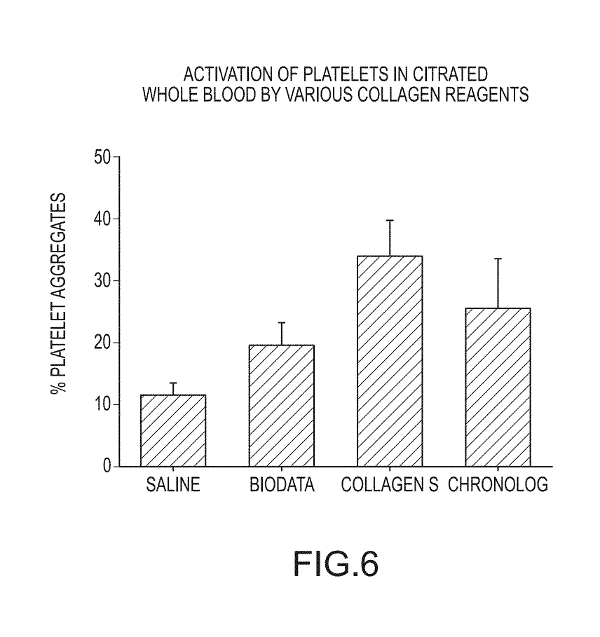

The ability of the various collagen reagents to induce platelet activation was assessed using whole blood flow cytometry. Platelet activation was assessed in terms of two parameters: P-selectin expression and formation of platelet aggregates. In this assay, platelet aggregates are defined as CD61(+) events with a size (forward angle light scatter) greater than that of the unaggregated platelet population. All collagen reagents were able to induce P-selectin expression on the platelet surface (FIG. 5) although the Bio/Data collagen was much less effective compared to the other reagents. A similar trend was observed with the formation of platelet aggregates in whole blood (FIG. 6).

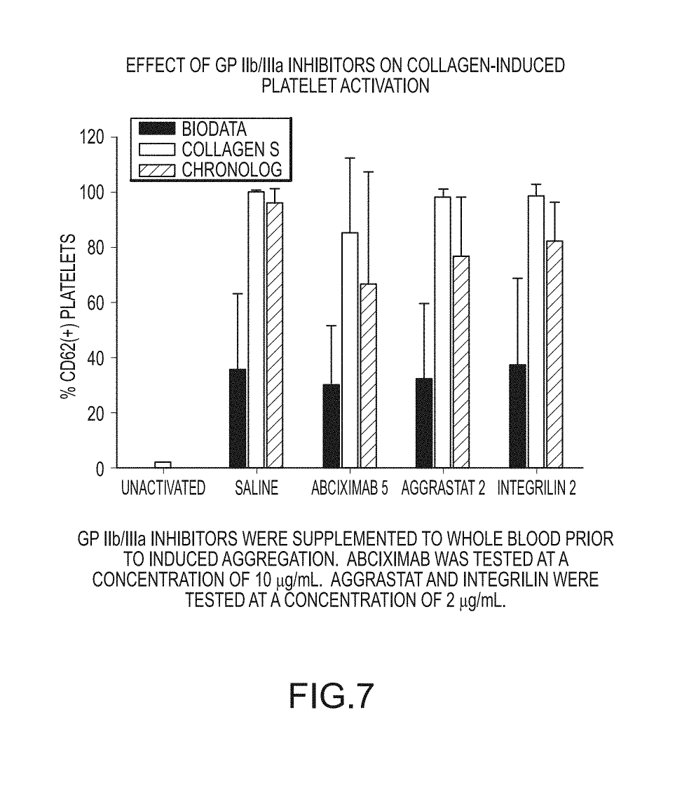

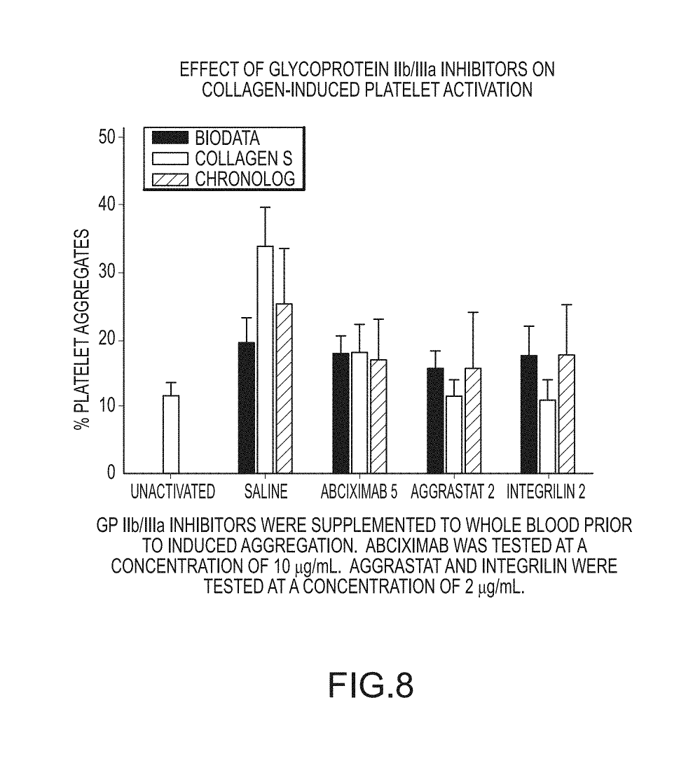

Supplementation of GP IIb/IIIa inhibitors to whole blood prior to activation also had little effect of collagen-induced P-selectin expression, but did prevent platelet aggregate formation (FIGS. 7 and 8).

### Example 2: Evaluate the Use of Synthetic Collagen to Detect the Anti-Platelet Activity of Ticagrelor, Cilostazol and Abciximab in Normal Human Platelet Rich Plasma

### Materials: Anti-Platelet Drugs

Ticagrelor (Brilinta®, Astra-Zeneca, London, UK; lot AL0153, expiration 02/14) was obtained as 90 mg tablets from the Loyola University Health System inpatient pharmacy. Tablets were ground using a mortar and pestle and subsequently dissolved in DMSO at a concentration of 10 mg/mL. The stock solution was diluted in deionized water to make working solutions of 0.5, 0.1 and 0.05 mg/mL.

Cilostazol (Pletal®, Otsuka Laboratories, Tokushima, Japan; lot 0B91M) was obtained as a powder. Cilostazol was dissolved in DMSO to make a stock solution of 5 mM. The stock solution was diluted in deionized water to make working solutions of 250, 125 and 50 μM.

Abciximab (ReoPro®, Eli-Lilly, Indianapolis, Ind.; lot 12D09AA, expiration 05/15) was obtained as a 2 mg/mL solution which was diluted in physiologic saline to make working solutions of 12.5, 25 and 50 μg/mL.

### Materials: Platelet Agonists

ADP was obtained from Bio/Data Corporation, Horsham, Pa. Each vial was reconstituted with 1 mL of deionized water to make a 100 μM working solution. The final concentration of ADP in the aggregation cuvette was 10 μM.

Arachidonic acid was obtained from Bio/Data Corporation, Horsham, Pa. Each vial was reconstituted with 0.5 mL deionized water to make a 5 mg/mL working solution. The final concentration of arachidonic acid in the aggregation cuvette was 500 μg/mL.

Collagen was obtained from Bio/Data Corporation, Horsham, Pa. Each vial was reconstituted with 0.5 mL deionized water to make a 1.9 mg/mL working solution. The final concentration of Bio/Data collagen in the aggregation cuvette was 190 μg/mL.

Collagen was obtained from Chrono Log Corporation, Havertown, Pa. as a 1 mg/mL solution. A working solution of 100 μg/mL was made by dilution with physiologic saline. The final concentration of Chrono Log collagen in the aggregation cuvette was 10 μg/mL.

Synthetic collagen was provided by JNC Corporation, Yokohama, Japan at working concentrations of 80, 160, 320 and 640 ng/mL. The final concentrations of synthetic collagen in the aggregation cuvette were 64, 32, 16 and 8 ng/mL.

### Methods: Blood Collection

Approval for the collection of whole blood from healthy human volunteers was granted from the Institutional Review Board of the Health Sciences Division of Loyola University Chicago. Whole blood was drawn using a double syringe technique from the antecubital vein and anticoagulated by the addition of 1 part 3.2% sodium citrate to (9 parts blood to 1 part citrate). Citrated blood was centrifuged at 80×g at room temperature for 15 minutes to make platelet rich plasma (PRP). Supernatant PRP was collected and kept at room temperature in capped tubes. The remaining citrated blood was recentrifuged at 1,100×g for 15 minutes to make platelet poor plasma (PPP). The platelet count of the PRP was determined using an ICHOR II Analyzer, Helena Laboratories, Beaumont, Tex. Platelet count in the PRP was adjusted to 250,000-300,000/μl by the addition of homologous PPP.

Six blood donors were used for this study. Each was drawn on separate days for the ‘non-aspirinized’ portions of the protocol. For the ‘non-aspirinized’ portion of the protocol, one donor (CS) was excluded from the final analysis as the aggregation responses were markedly different from that of the other 5 donors.

### Methods: Platelet Aggregation

Platelet aggregation was measured using a PAP 8E platelet aggregometer (Bio/Data). Each well was blanked using PPP. 25 μl of saline or antiplatelet drug and 200 μl of PRP were added to cuvettes containing magnetic stir bars and incubated for three minutes to equilibrate the sample to 37° C. 25 μl of agonist was added to each cuvette and the aggregation profile was monitored until a plateau was achieved. Results were tabulated in terms of maximal aggregation level. Under some reaction conditions, reversible aggregation was observed. This was most commonly observed with ADP and arachidonic acid-induced aggregation in the presence of ticagrelor or cilostazol. Final aggregation levels were also tabulated.

### Results:

### Ticagrelor

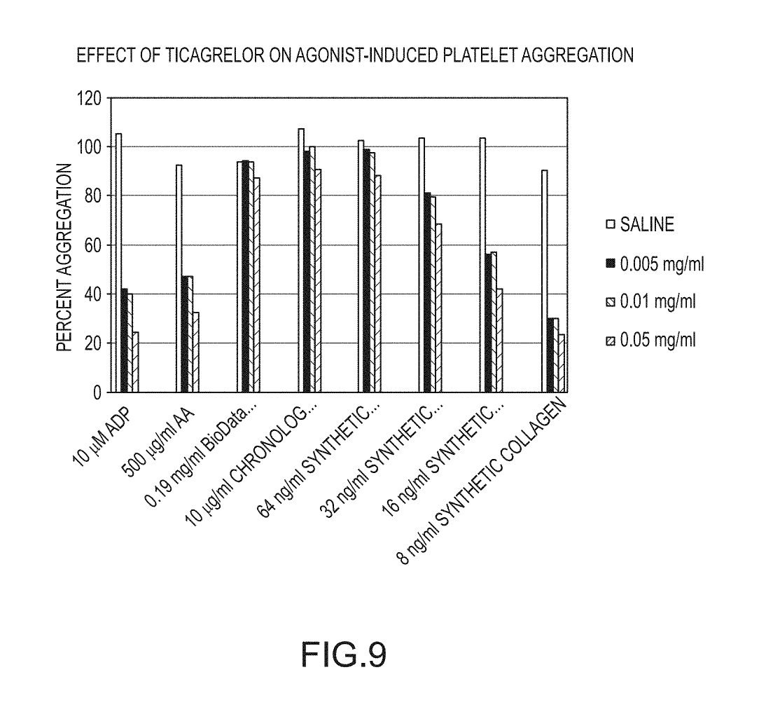

In non-aspirinized plasma, ADP-induced aggregation was strongly inhibited (FIG. 9). Arachidonic acid-induced aggregation was inhibited to a similar extent. Neither Bio/Data collagen, Chrono Log collagen nor 64 ng/mL synthetic collagen was markedly impacted by ticagrelor. The antiplatelet effects of Ticagrelor® could be identified when aggregation was induced by synthetic collagen at concentrations of 32 ng/mL and lower. The sensitivity for ticagrelor detection appeared to increase with decreasing synthetic collagen concentration.

### Cilostazol

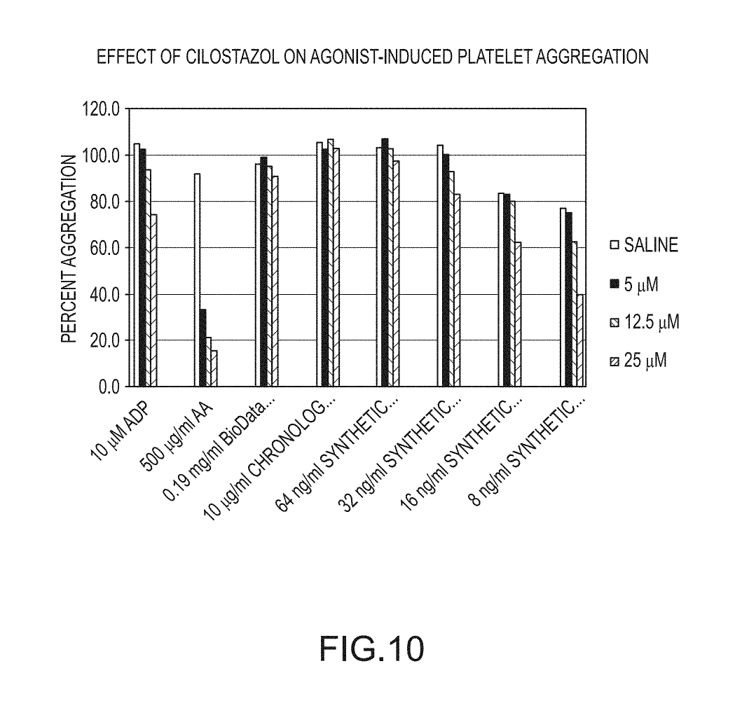

In non-aspirinized plasma, the most marked effect of cilostazol was on arachidonic acid-induced aggregation (FIG. 10), where aggregation levels of ˜20% were observed at concentrations ≥12.5 μM (vs. 95% in the absence of cilostazol). ADP-induced aggregation was minimally affected (˜30% inhibition at 25 μM). Cilostazol at concentrations up to 25 μM did not inhibit aggregation induced by Bio/Data collagen, Chrono Log collagen or the 64 ng/mL concentration of synthetic collagen. At lower concentrations of synthetic collagen, the anti-platelet effect of higher concentrations of cilostazol could be observed.

### Abciximab

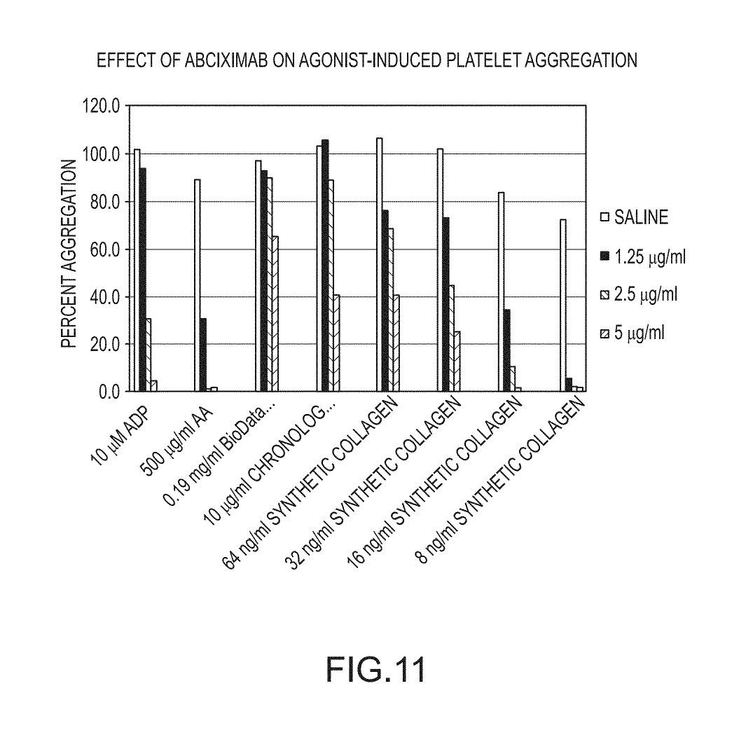

In non-aspirinized plasma, abciximab produced a concentration-dependent inhibition of agonist-induced aggregation over the concentration range tested (1.25-5 μg/mL) (FIG. 11). Arachidonic acid and synthetic collagen (8 and 16 ng/mL) were the most sensitive for detecting the presence of abciximab. Inhibition of Bio/Data and Chrono Log collagen-induced aggregation was only observed at the 5 μg/mL concentration.

### Discussion:

ADP, arachidonic acid and collagen are commonly used agonists to study platelet function. Ticagrelor inhibited aggregation induced by ADP and arachidonic acid, but had little effect on collagen-induced aggregation. Cilostazol strongly inhibited arachidonic acid-induced aggregation and produced a weaker, concentration-dependent inhibition of ADP-induced aggregation. Collagen-induced aggregation was unaffected by cilostazol. Abciximab inhibited aggregation induced by ADP, arachidonic acid and collagen, though ADP and arachidonic acid were more sensitive.

The synthetic collagen reagent was tested at concentrations ranging from 8 to 64 ng/mL. Aggregation induced by the 64 ng/mL concentration of the synthetic collagen was comparable to that of the Bio/Data and Chrono Log collagen reagents in that there was minimal effect of ticagrelor or cilostazol on the aggregation response. Aggregation induced by collagen or the 64 ng/mL synthetic collagen was inhibited by abciximab to a comparable degree. At lower concentrations of synthetic collagen, the antiplatelet effect of ticagrelor, cilostazol and abciximab were readily apparent.

1. A platelet aggregation test for determining an individual's anti-platelet medication sensitivity status, when the individual is on an anti-platelet medication therapy, the assay comprising the use of synthetic collagen at a final concentration in the platelet aggregation test from about 2 ng/mL to about 500 ng/mL.

2. The aggregation test of claim 1, wherein the test comprises light transmission aggregometry assays.

3. The method of claim 1, wherein in the synthetic collagen comprises a polypeptide having a peptide fragment represented by the formula (I)
-(Pro-X-Gly)ₙ (I)
wherein X represents Hyp; and n represents an integer of from 20 to 250.
